(19) United States
(12) Patent Application Publication
(10) Pub. No.: US 2007/0044539 A1
(43) Pub. Date: Mar. 1, 2007

(54) System and method for visual representation of a catastrophic event and coordination of response

(76) Inventors: Bryan Sabol, Vaughn, WA (US); Miles N. Moore, Carlsbad, CA (US); J. P. Pollak, Carlsbad, CA (US); Andrew Bowker, (US); David Korz, Cupertino, CA (US)

Correspondence Address: COOLEY GODWARD KRONISH LLP, 3000 EL CAMINO REAL, 5 PALO ALTO SQUARE, PALO ALTO, CA, 94306, US

(21) Appl. No.: 11/366,223

(22) Filed: Mar. 1, 2006

Related U.S. Application Data
(60) Provisional application No. 60/657,787, filed on Mar. 1, 2005.

(51) Int. Cl. G08B 23/00; G01N 7/00; G01N 11/00; G08B 21/00; G01N 33/497; G01N 33/487

(52) U.S. Cl. 73/19.01; 340/500; 340/540; 73/23.2; 73/53.01

(57) ABSTRACT

A method for monitoring an environment for threat conditions potentially related to a catastrophic event is disclosed herein. The method includes determining baseline levels of one or more environmental agents in an area based upon first measurement data received from a sensor network. The method further includes establishing one or more threshold levels relative to the baseline levels. Second measurement data received from the sensor network is then processed with respect to the one or more baseline levels. The method also includes identifying at least one threat based upon the processing of the second sensor measurement data. The identified threat is then assesses and prioritized.

### CROSS-REFERENCE TO RELATED APPLICATIONS

This application claims priority under 35 U.S.C. §119(e) to U.S. provisional This application claims priority under 35 U.S.C. §119(e) to U.S. provisional application Ser. No. 60/657,787, entitled SYSTEM AND METHOD FOR VISUAL REPRESENTATION OF A CATASTROPHIC EVENT AND COORDINATION OF RESPONSE, filed Mar. 1, 2005.

### FIELD OF THE INVENTION

The present invention generally relates to the detection of catastrophic events precipitated by, for example, chemical, nuclear and/or biological attacks or other hazardous incidents, and to the management of response to such events. More particularly, the present invention relates to a system and method for detecting and providing a visual representation of the environmental consequences of such attacks and for facilitating management of a coordinated response.

### BACKGROUND OF THE INVENTION

There is currently widespread concern about the potential for the nefarious use and dissemination of chemicals, radiation and biological agents. Such dissemination may result from, for example, terrorist activity or as a consequence of an industrial accident. In either case, minimization of damage, injuries and possible loss of life requires location and identification of the applicable hazard and initiation of an appropriate response, such as issuance of alerts to responsible “first responders” and other authorities.

Various systems and devices exist for detecting chemical and biological agents and radiation. For example, sensor networks are capable of detecting the presence of radiological devices (“dirty bombs”), as well as certain chemical and biological agents. Unfortunately, existing detection and alert processes and systems are primarily focused upon the initial detection of a hazardous or terrorist event, and are less concerned with facilitating coordinated responses to such events. Moreover, these existing processes often rely upon labor-intensive information gathering and information filtering techniques, which often precludes real-time threat evaluation and response coordination.

The recent terrorist events in the United States and elsewhere have highlighted the need to enable rapid and accurate evaluation of emergency events and to improve the coordination among first responders. In this regard management of information pertinent to emergency events has been challenging as a consequence of the diverse nature of the information received, and the need to nearly immediately evaluate and analyze such information. Yet other difficulties are involved in enabling emergency responders, government officials and other relevant parties to have timely access to the information most relevant to them.

### SUMMARY OF THE INVENTION

In summary, the present invention relates in one aspect to a method for monitoring an environment for threat conditions potentially related to a catastrophic event. The method includes determining baseline levels of one or more environmental agents in an area based upon first measurement data received from a sensor network. The method further includes establishing one or more threshold levels relative to the baseline levels. Second measurement data received from the sensor network is then processed with respect to the one or more baseline levels. The method also includes identifying at least one threat based upon the processing of the second sensor measurement data. The identified threat is then assesses and prioritized.

The present invention also relates to a system for detecting an event having environmental consequences. The system includes a plurality of sensors capable of detecting one or more environmental agents. The system further includes a plurality of sensor modules, wherein each of the plurality of sensor modules is capable of receiving data produced by at least one of the plurality of sensors. A central server is in communication with one or more of the plurality of sensor modules, and is configured to generate an alert indicative of occurrence of the event based upon information received from the one or more of the plurality of sensor modules.

### BRIEF DESCRIPTION OF THE DRAWINGS

For a better understanding of the nature of the features of the invention, reference should be made to the following detailed description taken in conjunction with the accompanying drawings, in which:

FIG. 1A illustratively represents a simplified embodiment of the catastrophic event detection system of the present invention.

FIG. 1B illustratively represents an embodiment a Visual Intelisense™ system of the present invention characterized by a distributed, clustered topology.

FIG. 2 is a flow diagram which illustratively represents an exemplary sequence of operations performed by the Visual Intelisense™ system in accordance with an embodiment of the invention.

FIG. 3 provides a block diagrammatic representation of the structure of a server computer configured to execute the Intelisense™ server.

FIG. 4 illustrates a high-level representation of the data flow through an exemplary sensor module.

FIG. 5 illustrates a high-level representation of exemplary data flow between the connection interface, interface handlers and database of a server within the system of the invention.

FIG. 6 is a flow diagram which illustrates the manner in which threat information is collectively processed within a server by the threat identification module, threat assessment module and the prioritization module.

FIG. 7 illustrates the general structure of, and relationship between, exemplary object classes corresponding to a sensor module and to a server.

FIG. 8 illustrates a screen shot of a user interface of the Map Window type.

FIG. 9 illustrates a screen shot of a user interface of the Event Window type.

FIG. 10 depicts a screen shot of a user interface or the Administration Window type.

FIG. 11 shows another screen shot of an Administration Window which illustrates the manner in which the settings of sensors may be adjusted.

FIGS. 12-20 illustratively represent the manner in which the inventive Visual Intelisense™ system may be utilized to monitor large-scale environments, detect emergency events, alert appropriate first responders, and potentially activate automated systems.

FIG. 21 provides an illustration of visualization method which comprises dividing a large scale map into multiple, smaller maps which are tiled to fit the display of the Intelisense™ Client.

FIG. 22 is a screen shot of a user interface of a view of intermediate scope, through which may be displayed a single, entire location, such as a city or county, fully within the browser window.

FIG. 23 is a screen shot of a user interface of a view achieved using various “zooming” functions, which permit a user to zoom into a location to an extent limited only by the resolution of the underlying map.

FIG. 24 depicts a screen shot of an exemplary Layers dialog.

FIG. 25 is a screen shot illustrating an Advanced view of the Layers dialog.

FIG. 26 is a screen shot of a History Viewer interface illustrating a first mode of historical data visualization.

FIG. 27 shows a screen shot of a user interface which illustrates a second mode of historical data visualization.

FIGS. 28-48 are screen shots of exemplary user interfaces presented in connection with operation of a specific implementation of the Visual Intelisense™ system of the present invention.

### DETAILED DESCRIPTION OF THE INVENTION

### System Overview

The present invention is directed to a novel system disposed for detecting, visualizing and enabling response to sudden, catastrophic events such as those precipitated by terrorist attacks or incidents involving hazardous materials. The system of the present invention is believed to be particularly useful in connection with managing responses to catastrophic events occurring in urban centers, military installations, and other high profile and/or densely inhabited environments. Embodiments of the innovative system are capable of being integrated with existing, deployed chemical, radiation and biological sensors or sensor networks.

A number of aspects of embodiments of the Visual Intelisense™ system have been designed to support first responders and other emergency operations personnel. In particular, the detection function of the Visual Intelisense™ system provides substantially constant, automated monitoring of collected environmental information in order to discern the existence of terrorist activities or other catastrophes, including the detonation of radiological devices and the release of chemical or biological agents. A second or “alert” function of the Visual Intelisense™ system initiates substantially instant notification of specified personnel following detection of an emergency event via a variety of end-user devices (e.g., personal computers via email, pagers, cellular phones, wireless PDAs). The Visual Intelisense™ system also facilitates near real-time situational awareness (e.g., containment status) on the basis of the received sensor data. The Visual Intelisense™ system is also capable of interfacing with existing emergency response systems and can be pre-configured to activate these systems and pass along details of the applicable emergency event. Finally, first responders which have been notified by the inventive system and arrive at the scene of the event may utilize tracking devices which send their position, status and other critical information back to the system. Iconic representations of this information may be overlaid by the system on a map directed to the event in order to enable responsible authorities to possess full situation awareness and be cognizant of potential economic impact. This awareness enables responsible authorities to communicate with and command appropriate groups of responders in order to coordinate their actions.

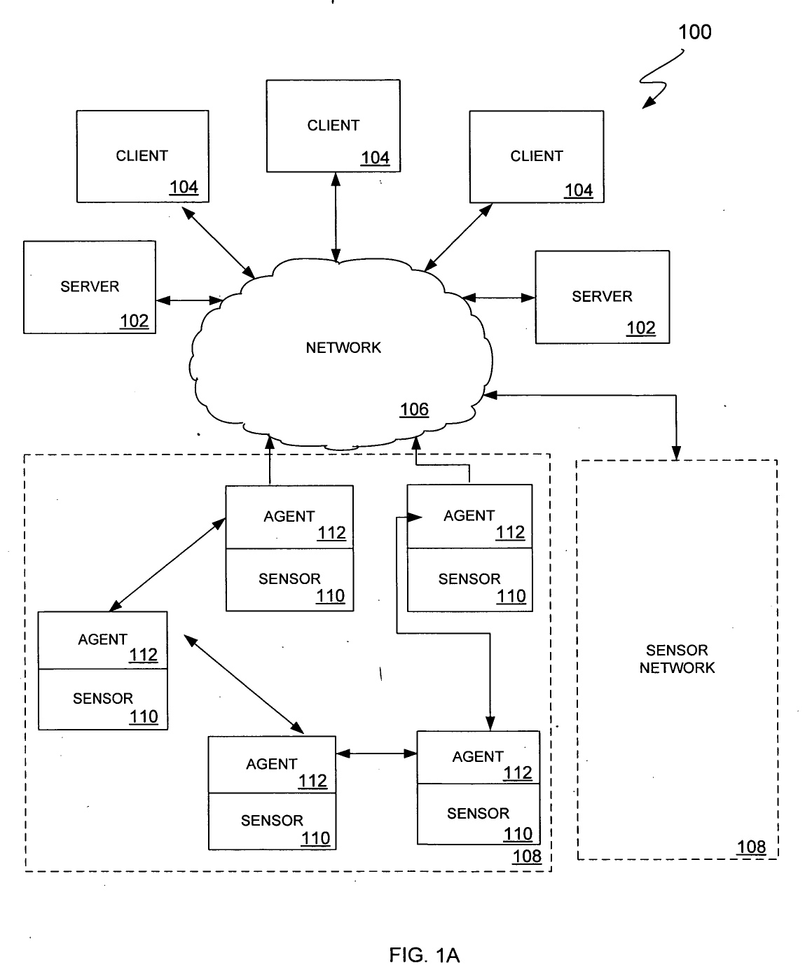

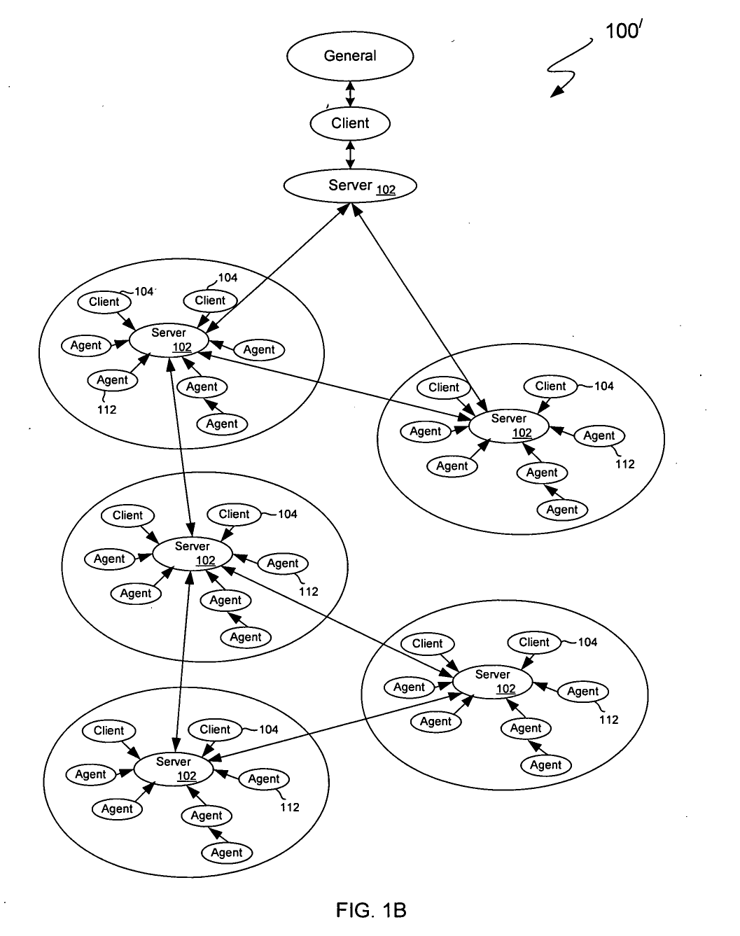

FIG. 1A illustratively represents a simplified embodiment of the catastrophic event detection system 100 of the present invention. As shown, the system 100 includes one or more servers 102 in network communication with a plurality of clients 104. Although for clarity of presentation the simplified embodiment of FIG. 1A depicts only a limited number of clients 104 and servers 102, other embodiments may include relatively larger number of clients and servers in communication over a communication network 106 (e.g., the Internet).

In what follows the system 100 may be interchangeably referred to as the “Visual Intelisense™” system, or simply as “Visual Intelisense™”. In addition, the servers 102 the system 100 may interchangeably be referred to as “Visual Intelisense™ servers” or “Intelisense™ servers”, and the clients 104 may be referred to as “Visual Intelisense™ clients” or “Intelisense™ clients”.

The Intelisense™ servers 102 are configured to receive data streamed from one or more sensor networks 108 deployed within geographic regions of interest (e.g., a densely-populated urban center or military installation). Each sensor network 108 includes a plurality of sensors 110 coupled to corresponding sensor modules or “agents” 112. As is described below, each sensor agent 112 controls the relaying of data from a corresponding sensor 110 to one or more modules (described below) executed by a server 102. Consistent with one aspect of the invention, the parameters of the data relaying function effected by the sensor agents 112 (e.g., frequency of reporting) may be controlled by users of the Intelisense™ clients 104. Although in the embodiment of FIG. 1A each sensor agent 112 is depicted as being co-located with a corresponding sensor 110, in other embodiments the sensor agents 112 may be executed by the Intelisense™ server 102 such that each is configured to receive and process “raw” data from an associated sensor 110.

In the exemplary embodiment each sensor 110 comprises a toxic chemical sensor, a radiation sensor, a biological sensor, or some other form of environmental sensor. The Visual Intelisense™ system is disposed to operate with virtually any conventional sensor having an electronic interface, and does not require the deployment of specialized or proprietary sensors or detectors. However, in other embodiments one or more of the sensors 110 may correspond to individuals located near the scene of an emergency event. In such instances communication with the Intelisense™ server 102 may be effected via conventional radio or telephone networks.

Each Intelisense™ client 104 comprises a software module used to administer the Visual Intelisense™ system and to view maps, sensors, and various overlays based upon information delivered from one of the Intelisense™ servers 102. In the exemplary embodiment each Intelisense™ client 104 is created in the Microsoft .NET framework, and may be executed by conventional computer platforms within command and control centers as well as by mobile computing devices (e.g., personal digital assistances) distributed to emergency field personnel.

As is described below, the Visual Intelisense™ system is designed to provides superior command and control to first responders and emergency operations centers with real-time software that networks existing sensors and other detectors into an intelligent sensor network. In one exemplary embodiment the Visual Intelisense™ system constantly and automatically scans multiple areas against terrorist attacks and other sudden, catastrophic events that may occur in geographic regions of interest. This enables immediate generation of alerts to proper authorities, agencies, and healthcare facilities so as to establish situation awareness, which may minimize loss of life and facilitate coordination of response. All of the relevant threat-related and other information can be overlaid on a map of the affected area to help illustrate the nature of the event and speed the decision-making process by providing critical information in an easy-to-digest format for display via an Intelisense™ client.

FIG. 1B illustratively represents an embodiment the Visual Intelisense™ system 100′ of the present invention characterized by a distributed, clustered topology. As shown, the system 100′ includes a plurality of Intelisense™ servers 102 in networked communication with each other. In addition, each Intelisense™ server 102 is disposed to receive measurement data from, and provide configuration instructions to, a number of sensor agents 112 respectively associated with a corresponding number of sensors 110 (not shown for purposes of clarity). Each Intelisense™ server 102 is also designed for networked communication with a set of Intelisense™ clients 104.

### Process Flow for Threat Detection, Alerting and Response Enablement

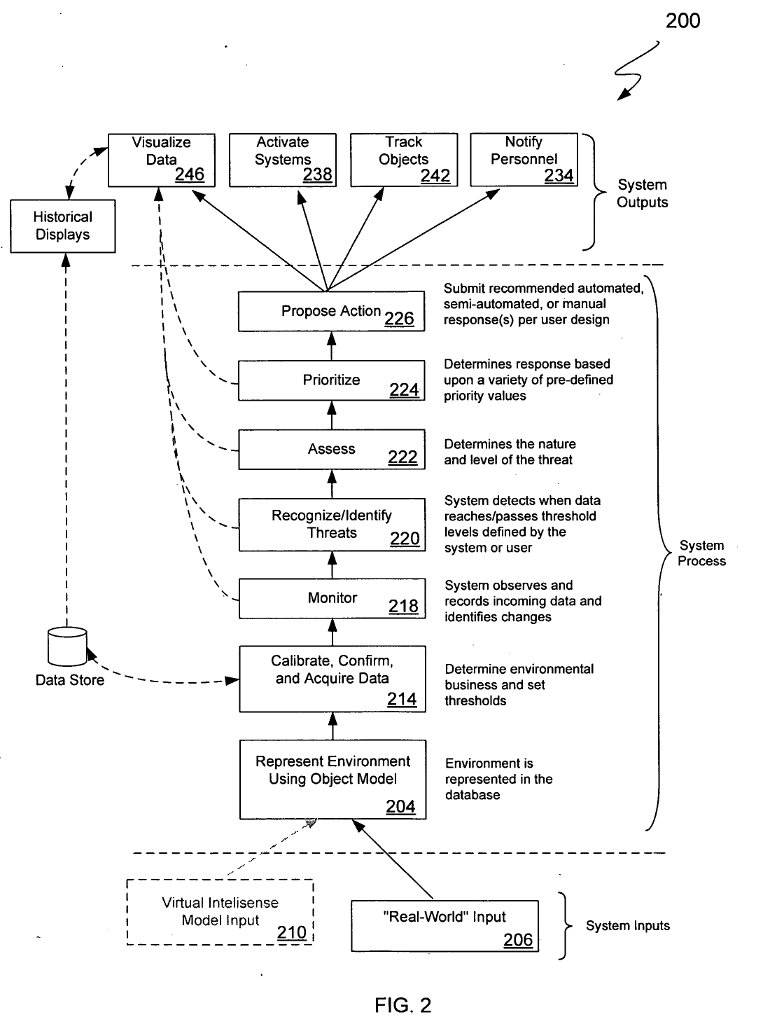

Turning now to FIG. 2, a flow diagram is provided which illustratively represents an exemplary sequence of operations 200 performed by the Visual Intelisense™ system 100 in accordance with an embodiment of the invention. Prior to initiating operation of the system 100, an object-oriented representation of the environment being monitored, as well as of the applicable monitoring and response infrastructure, is created and stored within or in association with one or more of the Intelisense™ servers 102 (stage 204). Although an example of a possible set of objects useful in developing this object-oriented environmental model is provided below, in typical implementations the model will include objects or “agents” representative of, for example, monitoring and response infrastructure such as individual sensors, sensor measurements, alerts generated based upon the sensor measurements, response teams and their status, maps and events generated by the model objects.

As shown in FIG. 2, the environmental object model is of course influenced by “real-world” input 206 corresponding to, for example, changes in the types of sensors deployed or their respective locations, actual sensor measurements, events, and changes in the location or composition of response teams. In short, any material change in the status of the deployed monitoring or response infrastructure will typically be reflected as a corresponding change within the object-oriented environmental model maintained by one or more of the servers 102. In addition, the monitoring and response infrastructure represented by the environmental object model may be modified on the basis of data obtained from simulated intentional (e.g., terrorist) attacks and accidental events occurring within the monitored environment (stage 210).

Referring again to FIG. 2, in an initial operative phase 214 of the system 100 the sensor networks 108 and any other deployed detectors acquire data in order to determine baseline environmental levels of radiation and/or of any chemical or biological agents being monitored. During this stage thresholds may also be set relative to the baseline levels in order to define unsafe concentrations or levels of the monitored environmental conditions.

During the remaining phases of operation of the system 100, measurement data streamed from the sensor network 108 to one or more of the servers 102 is compared against various sets of rules within a rules engine defined by a system administrator or other user. These phases may be characterized as a monitor phase 218, a recognize/identify threats phase 220, an assessment phase 222, a prioritization phase 224 and a propose action phase 226. These phases will generally operate in the sequence depicted in FIG. 2 with respect to sensor measurement data acquired contemporaneously during a given time period. However, since such measurement data will typically be received by the applicable server 102 in a continuous stream, it is also the case that the respective phases will operate in parallel at any given point in time (i.e., each being engaged in processing measurement data acquired during a different time period).

Referring again to FIG. 2, the processing effected by the applicable server 102 during the monitor phase 218 involves observing and recording incoming measurement data from the sensor networks 108 and identifying changes relative to thresholds set during the previous phase 214. The types of environmental changes detected and reported during execution of the monitor phase 218 will depend on the nature of the sensors 110 in the respective networks 108. However, at the most fundamental level, the sensor agents 112 connect to their associated sensors 110, collect ambient sensor measurement data, and pass the collected data through the sensor network 108 to the server 102. It is noted that each sensor agent 112 may represent a virtual sensor rather than a physical sensor (e.g., during performance of a simulation), but for the sake of the present discussion it is assumed that the sensor agents 112 receive data from, and are representative of, actual physical sensors 110. Under normal circumstances, the agents 112 stream measurement data collected by a corresponding sensor 110 to the server 102 in accordance with a user-defined or default sampling frequency. For example, when battery-operated sensors are utilized it becomes important to conserve power by, for example, minimizing the power expended to communicate measurement data (“readings”) from such sensors to the server 102. Accordingly, in such cases each agent 112 associated with such a sensor may set a periodic sampling rate, buffer a number of the readings provided by the sensor, and then instruct that a batch of readings be transmitted at once in order to minimize power drain. If a reading hits a pre-defined “threshold” level, the applicable agent 112 can immediately change its behavior to increase the applicable sampling frequency and begin continuously streaming the readings to the server 102 rather than continue to buffer the readings received.

In one embodiment the recognize/identify threats phase 220 involves detecting when readings from specific sensors exceed the threshold levels set during phase 214. Although this embodiment corresponds to perhaps the most straightforward implementation of the phase 220, substantially more complex implementations of the threat identification process have also been contemplated and are summarized below.

Consistent with one aspect of the invention, implementation of the assessment phase 222 is facilitated by associating each sensor 110 with one or more different types of predefined profiles. For example, if a particular radiation sensor was associated with a “sensor profile” which characterized its sensor type as well as with a particular “region profile” (e.g., “Washington DC Subway”), a “scenario profile” could then be set in the rules engine which would be invoked if only the particular sensor detected a radiation level in excess of a predefined threshold only slightly above background levels. Of course, invocation of other scenario profiles could require that a larger number (e.g., 5 or more) radiation sensors in the same area all detect radiation levels exceeding a substantially higher threshold. In contrast, region profiles encompassing less sensitive areas could require that a similarly large number of sensors register readings exceeding even a higher threshold in order for the same or an analogous scenario profile to be invoked.

The prioritize phase 224 involves determining, in accordance with a set of predefined rules, an appropriate response to a threat identified during the preceding assessment phase 222. In one embodiment a plurality of predefined priority values are established prior to initiating operation of the system 100, and one of these is assigned during the prioritize phase 224 to each threat identified during the assessment phase 222. As a consequence, threats which have been identified as being serious in nature can be assigned a relatively high priority value and allocated appropriate amounts of system resources. For example, system processing priority may be given to threats that are identified as high priority. In addition, the visual representations provided to end users via clients 104 may be configured to display, filter, and/or sort threat or other event information by the priority accorded such information.

Execution of the propose action phase 226 results in automated, semi-automated or manual response recommendations being submitted to various entities in accordance with the applicable scenario profile. In the exemplary embodiment these various recommended responses are specified, using a rules engine, for each different scenario profile by users or administrators of the system 100. This effectively permits specification of responses to each different event, uniquely identified by differences in any combination of type of sensor, level of reading, threshold setting, region, and number of additional sensors in the same area reporting similar events. The proposed action for each situation typically includes one or more of (i) notifying personnel or facilities with regard to the nature of the event (stage 234) (e.g., by sending a low priority message to a single individual, as opposed to sending a high priority message to a group of high-ranking officials), (ii) sending appropriate codes to activate/deactivate automated systems (stage 238) (e.g., in order to shut down HVAC equipment, reroute traffic signals, reverse the direction of a subway train, or automatically close an entrances), (iii) track objects of interest (stage 242), and (iv) generate a multi-layered visual representation information pertaining to the event for display upon clients 104 (stage 246).

### Architecture of Intelisense™ Server

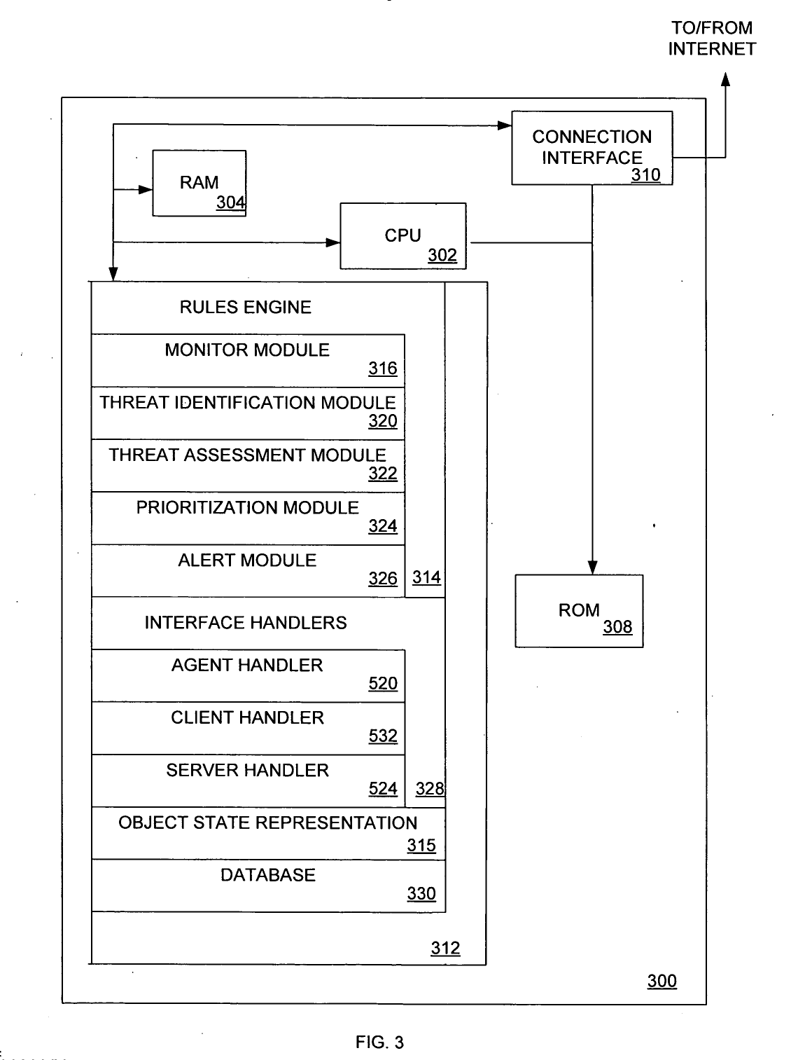

Turning now to FIG. 3, a block diagrammatic representation is provided of the structure of a server computer 300 configured to execute the Intelisense™ server 102. The server computer 300 includes a CPU 302 connected to RAM 304, ROM 308, a connection interface module 310 and secondary storage 312. Stored within secondary storage 312 are a set of software program modules which, when executed by the server computer 300, effect the functionality of the Intelisense server 102.

As shown in FIG. 3, secondary storage 312 includes a rules engine 314 comprised of a monitor module 316, a threat identification module 320, a threat assessment module 322, a prioritization module 324, and an alert module 326. In the exemplary embodiment the rules engine 314 implements the intelligence of the system 100, and maintains within secondary data storage 312 a representation 315 of the state of each object used in modeling the monitored environment. Secondary data storage 312 also includes a copy of the operating system for the server computer 300 (not shown). When effecting the functionality described herein, the CPU 302 loads into RAM 304 and executes one or more modules of the rules engine 314 or other program modules stored within secondary data storage 312.

Secondary data storage 312 also includes a database 330 which contains historical sensor measurement data and other information. In the exemplary embodiment the database 330 is accessed via interface handlers 328 in the manner described below. Storage of such historical sensor measurement data facilitates execution of rules which involve comparison of current values to historical measurements or statistics. Historical data from the database 330 may also be made available to clients 104 for historical reporting or charting.

In an exemplary embodiment the rules engine 314 comprises includes a fixed set of rules comprising the base knowledge framework inherent within the rule set. In general, the rules engine prescribes a process for comparing individual or sets of sensor values against defined threshold values. If the applicable threshold is exceeded, a threat may be identified and assessed and a corresponding alert may be created. The conditions for identifying, assessing and prioritizing a threat and creating an alert can be complex and involve the values of many sensors, the time of day, the location, historical value ranges, and a variable number of recent measurements. Rules may be added, deleted, and changed dynamically during runtime operation of the system 100.

During operation of the system 100, the CPU 302 executes the monitor module 316 during the monitor phase 218 in order to observe and record incoming measurement data from the sensor networks 108 and identifying changes relative to predefined thresholds. The CPU 302 executes the threat identification module 320 during the recognize/identify threats phase 220 and thereby detects when the recorded measurement data exceeds the predefined thresholds levels. During the assessment phase 222, the CPU 302 executes the threat assessment module 322 and invokes an appropriate scenario profile in view of the sensor profile and recorded measurement data associated with the applicable sensor. The CPU 302 executes the prioritization module 324 during the prioritize phase 224 in order to determine, in accordance with a set of predefined rules, an appropriate response to a threat identified during the preceding assessment phase 222. Finally, during the propose action phase 226 the CPU 302 executes the alert module 326 and submits automated, semi-automated or manual response recommendations to various entities in accordance with the applicable scenario profile.

### System Data Flow and Processing

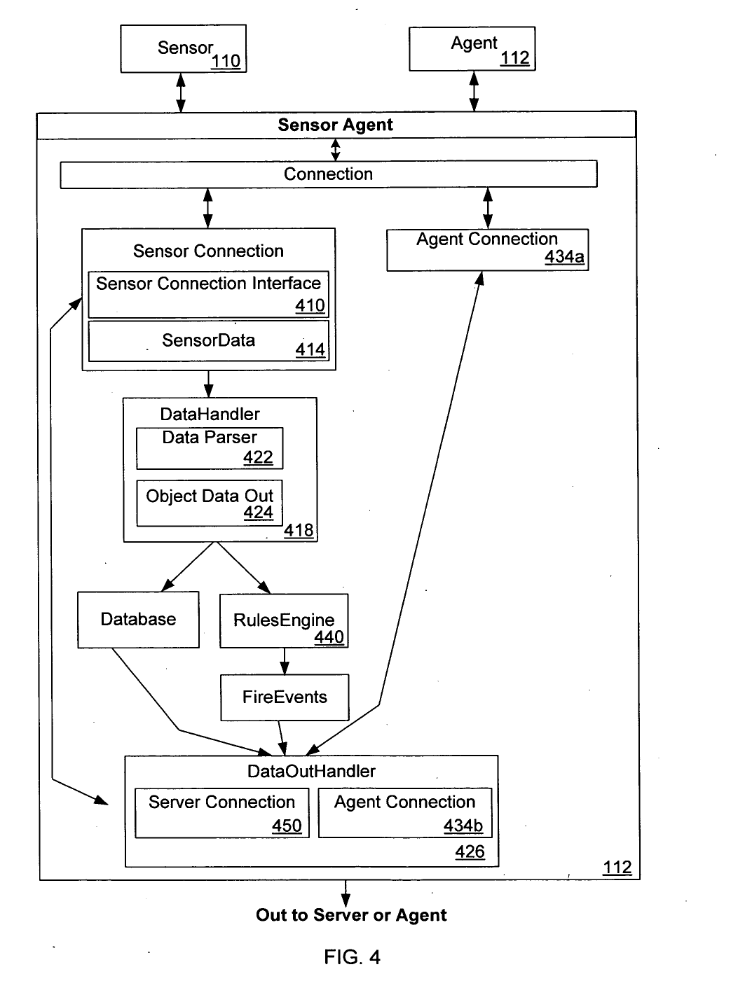

Referring now to FIG. 4, there is illustrated a high-level representation of the data flow through an exemplary sensor agent 112. Each sensor agent 112 will generally be configured to receive data either directly from its associated sensor 110 or from another sensor agent 112. If a given agent 112 is connected to another agent 112, then the given agent 112 simply routes the data of the other agent to either a server 102 or yet another agent to which the given agent 112 is connected. This topology allows sensor measurement and other data to be communicated over areas in which there may not exist a continuous communication link. Agents 112 which receive data from other agents 112 are generally unconcerned with the information content of the received data, and merely forward the received data for ultimate receipt by a server 102.

During operation of the system 100, a sensor 110 makes physical measurements and makes them available to its associated sensor agent 112 as digital data through either a serial or parallel interface. In mobile applications a sensor 110 may also provide location information, such as GPS coordinates, along with the physical measurement data. The physical measurement and other data generated by a sensor 110 is received by a “smart” sensor connection interface 410 of its associated sensor agent 112. The sensor connection interface may include a number of “plug-in” sensor drivers disposed to permit signals from a corresponding number of different types of sensors to be received and converted into an appropriate format expected by the sensor agent 112. To the extent it is desired to interface with a new type of sensor, a corresponding sensor driver may be added to the sensor connection interface. In the exemplary embodiment each sensor driver converts the measurement signal produced by a given sensor 110 into a sensor data object 414 comprised of the following attributes:

| | |
|---|---|
| ID | A unique tag assigned to the sensor when by the server, |
| | so that it can be identified |
| Location_X | latitude |
| Location_Y | longitude |
| Location_Z | altitude |
| Value | The value the sensor is sensing, (e.g., heat, light, |
| | nuclear, bio, chemical, etc.) |
| Time | The time at which the value was read |

As shown in FIG. 4, the sensor data objects 414 produced by the various sensor drivers are sent to a data handler module 418. In the exemplary embodiment the data handler module 418 includes a data parser 422 operative to produce a stream of sensor data objects 424 that can be sent to a server 102 or another agent 112 via a data out handler module 426. The stream 424 of sensor data objects are processed by server connection 430 of the data out handler module 426 when destined for a server 102, and are processed by an agent connection 434 when destined for another agent 112. The extent to which the stream 424 of sensor data objects is made available to the data out handler module 426 may be regulated in accordance with a set of rules stored within a sensor agent rules engine 440.

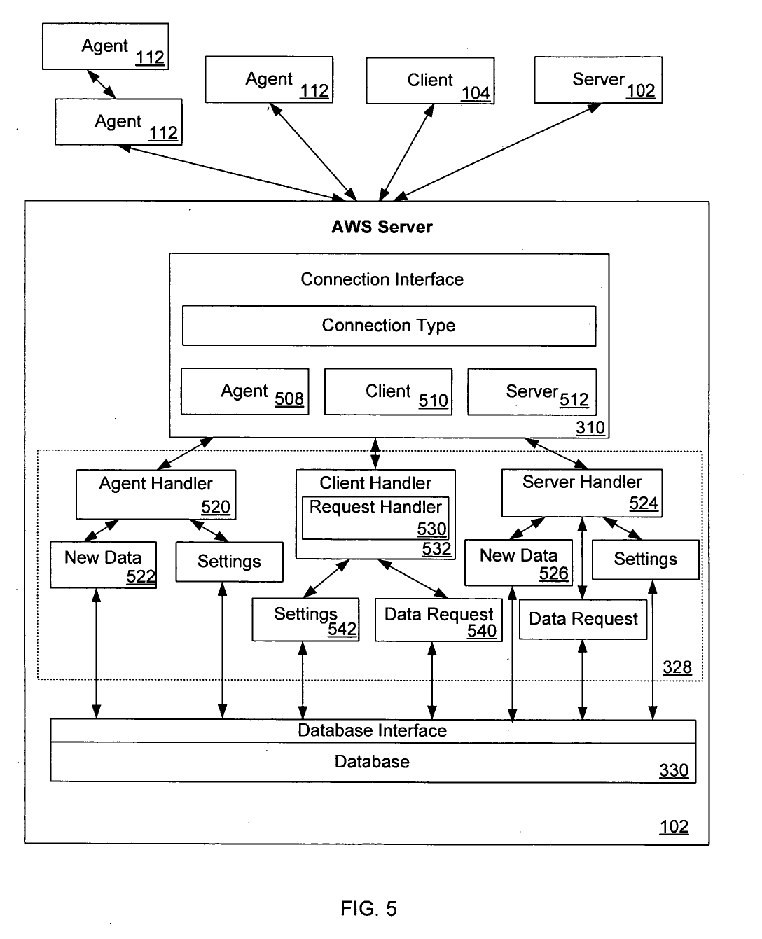

Referring now to FIG. 5, there is illustrated a high-level representation of exemplary data flow between the connection interface 310, interface handlers 328 and database 330 within a server 102. Each server 102 will generally be configured to receive data or requests from either sensor agents 112, another server 102 or clients 104. In addition, each server will typically also be capable of providing instructions or requests to sensor agents 112 and other servers 102, and of providing data for clients 104 to use for visual representations and other purposes. As shown, the connection interface 310 includes an agent connection 508, a client connection 510 and a server connection 512. The agent connection 508 handles the communication connections established by the server 102 with various agents 112, and passes incoming data from each such agent 112 to an agent data handler 520. In turn, the agent data handler 520 parses the incoming data and sends the resulting new data 522 to the database 330. The server connection 512 handles the communication connections established by the server 102 with other servers 102, and passes incoming data from each such other server 102 to a server data handler 524. In turn, the server data handler 524 parses the incoming data 526 and sends it to the database 330.

When a client 104 requests data from the server 102, the client connection 510 handles the request and initiates establishment of a communication session. The client connection 510 allows different types of clients 104 to connect to the server 102, including browser-based and application-based clients. The request received from the client 104 is passed by the client connection 510 to a request handler module 530 of a client handler 532. In the exemplary embodiment there exist two different types of requests which may be received and acted upon by the request handler module 530. Specifically, the request handler module 530 may be requested by a client 104 to (i) retrieve sensor measurement data 540 from the database provided by some or all of the sensors 110, or (ii) initiate the process of making changes to the configuration settings 542 of some or all of the sensors 110 and/or agents 112. In the case of (ii), the request handler module 530 initiates this process by changing the values of such settings stored within the database 330, which results in one or more messages being sent to the sensors 110 or agents 112 for which the change has been requested.

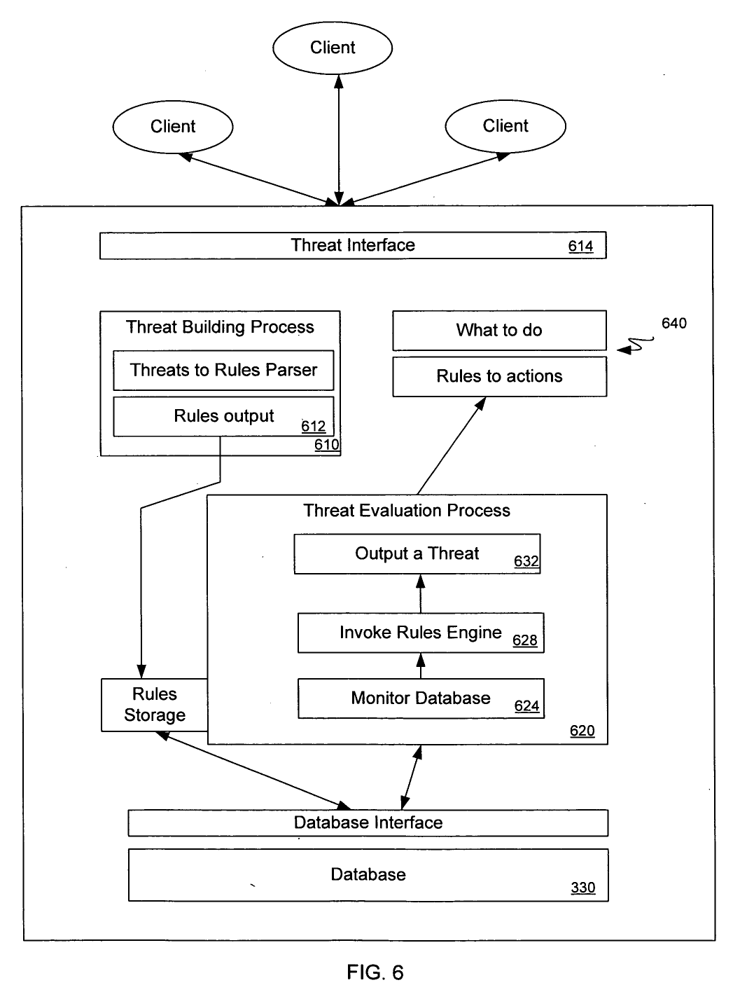

FIG. 6 is a flow diagram which illustrates the manner in which threat information is collectively processed within a server 102 by the threat identification module 320, threat assessment module 322 and the prioritization module 324. In the exemplary embodiment one or more clients 104 may initiate a threat building process 610 during a design phase preceding actual “runtime” operation of the applicable server 102 in order to create and otherwise define the rules 612 applicable to the runtime processing of threat information. The results of the runtime processing of threat-related information contribute to the visualization data and information ultimately provided to clients 104 by the applicable server 102. Communication between clients 104 and the threat processing elements of the server 102 is facilitated by a threat interface 614 during both the design phase and runtime operation.

As is indicated by FIG. 6, during runtime operation a threat evaluation process 620 is executed which involves monitoring 624 the database 330 for changes. Such monitoring 624 is responsive to each addition of information to the database by a user or agent 112. In particular, each time the data of a sensor 110 or a threshold is exceeded, the rules engine defined during the design phase is invoked 628. Although the present invention is not limited to use of any particular type of rules engine, in the exemplary embodiment the rules engine is implemented in with the syntax and other requirements of the Semantic Web Rule Language (SWRL). See, for example, http://www.daml.org/2004/04/swrl/. In the general case the syntax of the rules defined by the rules engine will be predicated upon the particular type of rules engine employed. The following provides an example of sample rule syntax:

if (Sensor.ID == x)

And if(Sensor.Value > x)
And if(Sensor.Location == x)
Then Do(Profile x)

And Start(Script x)

To the extent that invocation of the rules engine results in identification of a threat, the identified threat is output 632 to a threat prioritization process 640. In the exemplary embodiment threat prioritization is performed in accordance with standard weighting methods in the rules engine (e.g., the more important a threat factor, the higher its weight). The rules engine then uses the selected weights in order to determine which threats are of the highest priority. The rules engine also uses sensor data and profile data to set its rules. That is, the rules engine may be configured such that when the readings produced by sensor reach an identified level, a designated profile is activated.

### Types of Intelisense™ System Objects

As mentioned previously, an object model representation 315 (FIG. 3) of the environment being monitored and certain aspects of the response infrastructure is maintained by the rules engine 314. The object model representation 315 may be extended for new sensor types, networks, emergency response teams, and other tangible and intangible items as needed. A description of an exemplary set of types of objects which may potentially be included within such a representation is provided below; however, those skilled in the art will appreciate that additional or different objects may be utilized in other representations as necessary to appropriately reflect the environment being monitored or the response architecture.

Location

A location object represents a geographic area that is being monitored by sensors. Each location has a unique name. Co-ordinates for the location may be relative or in an absolute co-ordinate system. In the exemplary embodiment a number of different types of other objects may be associated to a particular location.

| Attribute | |
|---|---|
| Name | Description |
| mapname | String representing unique name of the map. |
| upperleft | Coordinate representing the upper left have corner of |
| | the map. Can be latitude/longitude, or any numeric value. |
| lowerleft | Coordinate representing the lower left have corner of |
| | the map. Can be latitude/longitude, or any numeric value. |
| upperright | coordinate representing the upper right have corner of |
| | the map. Can be latitude/longitude, or any numeric value. |
| lowerright | coordinate representing the lower right have corner of |
| | the map. Can be latitude/longitude, or any numeric value. |

In general, one pair of opposite corners should be given to determine location. All four corners need be specified only in cases where the applicable map is skewed by, for example, the angle at which the photo or satellite image corresponding to the map was taken.

Sensor Object

Sensor objects are characterized by a location and are generally representative of sensors disposed to produce measurements of a parameter of the surrounding physical environment, either substantially continuously or at fixed time intervals. Each sensor object is also characterized by a unique name. Sensor objects corresponding to physical sensors of the same or different types may be located in the same location. The location characterizing a sensor object may change between measurements in cases in which the sensor object is associated to a mobile physical sensor.

| | |
|---|---|
| Id | ID of the sensor |
| Displayname | Human readable name of the sensor |
| Mapname | Name of the map in which the set of sensors is |
| | located. |
| Location | The (x, y) location of the sensor relative to the |
| | map's origin. Editable for fixed sensors, but |
| | read-only for mobile agents. Sensors may also |
| | optionally belong to a Region Profile, whose |
| | inclusion would be determined by this location data. |
| Sensor Name | Name the sensor, independent of the unique ID the |
| | system allocates for it. |
| Sensor Type | Type of sensor |
| Manufacturer | Contact information for manufacturer of sensor or |
| | representative. |
| Detector | Specific type of detector that is contained in the |
| | sensor package (e.g., brand name and model) |
| Threshold | Defined threshold level of sensor. |
| Sampling | Frequency with which the sensor takes readings. |
| Frequency | |
| Reporting | Frequency with which the sensor transmits its |
| Frequency | collected data. The reporting frequency can be no |
| | shorter a time period than the sampling frequency. |
| Baseline | A moving average of the regularly sampled values. |
| Sensor Details | Read-only information concerning the sensor. |

Sensor Profile

Sensor Profiles greatly reduce the effort of manually controlling hundreds or thousands of sensors. Profiles let users group a specified set of the same type of sensors. Different types of profiles permit users to mix and match a variety of different sensors (based on sensor type, location, etc.), to obtain a very granular level of control and specificity.

| Attribute Name | Description |
|---|---|
| Id | ID of the sensor profile |
| Displayname | Human readable name of the sensor profile |
| Mapname | Name of the map in which the set of sensors is |
| | located. |
| Profile Name | Name the sensor, independent of the unique ID the |
| | system allocates for it. |
| Sensor Type | Type of sensor |
| Manufacturer | Contact information for manufacturer of sensor or |
| | representative. |
| Detector | Specific type of detector that is contained in the |
| | sensor package (e.g., brand name and model) |
| Threshold | Defined threshold level of sensor. |
| Sampling | Frequency with which the sensor takes readings. |
| Frequency | |
| Reporting | Frequency with which the sensor transmits its |
| Frequency | collected data. The reporting frequency can be no |
| | shorter a time period than the sampling frequency. |
| Baseline | Baseline parameters, such as type of moving average, |
| | length of the average, etc. |

Responder

Responder objects typically correspond to groups of first responders or other personnel. The responder object is intended to be sub-classed for specific types (e.g., police). Each type of sub-classes will generally include an additional set of unique attributes.

| Attribute Name | Description |
|---|---|
| Id | Unique id of the team |
| displayname | Human readable name of the responder team. |
| mapname | Name of the map in which the set of sensors is |
| | located. |
| location | The (x, y) location of the responder relative to |
| | the map's origin. |
| Status | Description of the status of the responder |

Alerts

Alert objects are declarations of a state of emergency or a set of conditions requiring response. An alert object is typically created by the rule engines by evaluating sensor measurements and other information. Alert objects are generally characterized by a state, a severity and a life cycle.

| Attribute Name | Description |
|---|---|
| Id | ID of the Alert |
| Type | Alert type, which are typically predefined in the |
| | system. |
| mapname | Name of the map the Alert is active in. |
| Area | A set of points representing a polygon the encloses |
| | the region in the map where the Alert is active. |
| Units | The units the area is measured in, e.g. feet or meters. |
| Cords | Area in real world co-ordinates. |
| coordsystem | Co-ordinate system for coords such as UTM or MGRS. |
| startime | Date and time in UTC when the Alert was started. |
| endtime | Date and time in UTC when the Alert ended. This will |
| | be null while the Alert is active. |
| State | State of the Alert, active and closed are defined by |
| | default. Additional states can be defined. |
| description | Description of the Alert. |
| Notes | An array of comments added to the Alert by the users. |

Events

Event objects are representative of changes occurring within the object-oriented representation of the Visual Intelisense™ system 100. For example, event objects will typically be created when another object is created or destroyed, or when an attribute of an object changes. In general, the creation of an event object may be caused by any other object, although specific event types may only be created by certain objects. For example, sensor measurement events may only be created by a sensor object or by an object representative of a sensor gateway. By definition, once an event has been created it does not change. In the exemplary embodiment all events have the following attributes:

| Attribute Name | Description |
|---|---|
| Id | Unique id |
| datetime | Date and time in UTC of the event. |
| Type | Type code of the event. |
| creator | JID of the event originator. |

### Exemplary Object Model Representation of Sensor Agent and Server

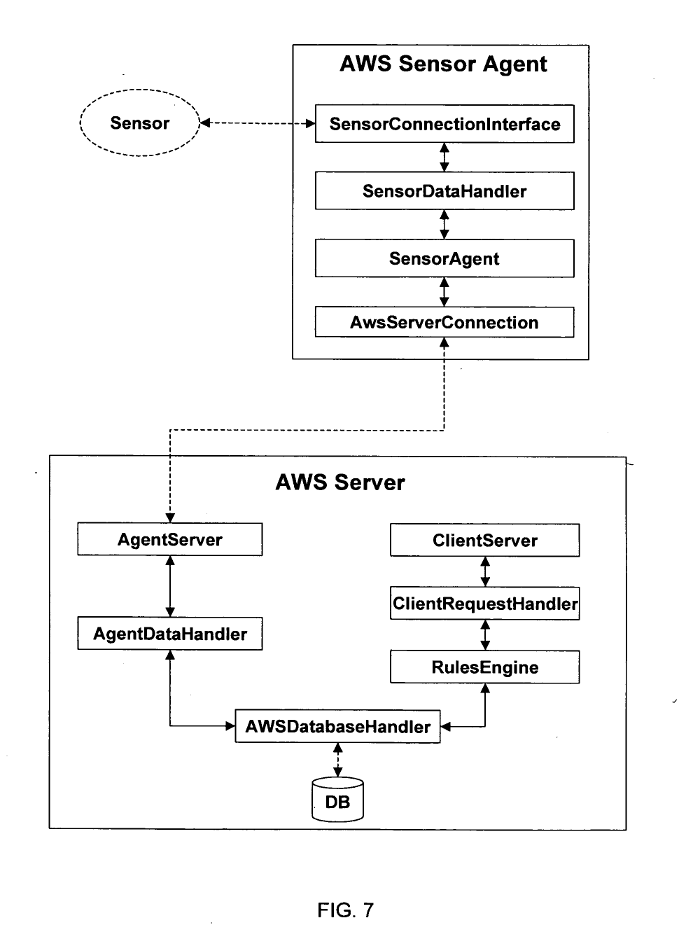

Referring now to FIG. 7, an illustration is provided of the general structure of, and relationship between, exemplary object classes corresponding to a sensor agent 112 and to a server 102. In particular, FIG. 7 illustrates an object class <AWS Sensor Agent> utilized in representation of the sensor agent and an object class <AWSServer> utilized in representation of a server 102.

As shown in FIG. 7, <AWS Sensor Agent> includes a SensorConnectionInterface disposed to accept data from one of many physical connection levels. The model is preferably of a plug-and-play design, whereby any sensor type with a software driver that outputs raw digital data can be plugged in to the agent. In an exemplary embodiment this interface may be of a number of different types, i.e., TCP, UDP, Serial, and Parallel:

<Sensor Connection Interface>
<TCP Connection>
<UDP Connection>
<Serial Connection>
<Parallel Connection>

The SensorConnectionInterface performs data error checking, and then passes the raw data (e.g., Sensor.Value=129, Sensor.Time=12:00) to SensorDataHandler. In the exemplary embodiment SensorDataHandler parses the raw data that came in though the SensorConnectionInterface. As mentioned above, substantially any type of sensor (e.g., GPS sensor, radiation sensor, etc.) may be accommodated by merely plugging in the sensor's software handler. SensorDataHandler reads the raw data, then generates a new sensor object that represents that sensor at the time the raw data was received by the SensorConnectionInterface.

The SensorAgent handles the sensor intelligence, such as heartbeats, turning sensor on/off, power consumption, instructing the sensor to read data, etc. SensorAgent performs calculations and routing of sensor data, but is left open for future development. SensorAgent also stores the intelligence set by the user from the sensor profile object and acts according to this object, by a rules engine implementation. SensorAgent can also connect to other agents to pass intelligence, and to route their information. SensorAgent receives the sensor object created by SensorDataHandler, then sends the sensor object to AwsServerConnection.

The AwsServerConnection is capable of implementing encryption/password protection with respect to sensor object and other information communicated over a server connection to an <AWSServer> object executed by a server 102. AwsServerConnection improves the robustness of such communication by preventing the loss of information in the event of temporary degradation of the applicable server connection. For example, if the server connection is lost, AwsServerConnection temporarily buffers the sensor objects to be transmitted from the <AWS Sensor Agent> object. Once the connection is re-established, the buffered sensor objects and other data are transmitted to the server 102. In particular, AwsServerConnection makes a connection to the <AWSServer> and sends the relevant sensor objects and other data.

In the exemplary embodiment all <AWS Sensor Agent> objects connect to the AgentServer of an <AWSServer> object. The AgentServer of an <AWSServer> object encrypts messages that are sent to <AWS Sensor Agent> objects and decrypts incoming sensor objects that are sent by <AWS Sensor Agent> objects through their respective AwsServerConnections. Once AgentServer receives raw data information from an <AWS Sensor Agent> object, it passes it to AgentDataHandler.

The AgentDataHandler feeds, in accordance with a load-balancing algorithm, sensor objects and other data to the RulesEngine. In general, AgentDataHandler converts the raw data received from the AgentDataHandler and “re-creates” sensor objects that can be stored in the database 330. It also identifies duplicate sensor data, and optimizes database usage by buffering the duplicate data until a new data value is received. The AgentDataHandler then converts the buffered data into a sensor object with a specific time span (e.g., 2pm-6pm) and sends it to the database 330. As shown in FIG. 7, the sensor objects re-created by the AgentDataHandler are passed to AWSDatabaseHandler.

The AWSDatabaseHandler handles the storage of, and access to, the sensor objects stored within the database 330 in an efficient manner. In the exemplary embodiment it stores sensor objects within the database 330 in a temporary memory to improve data access speed and reduce CPU load. In this way the AWSDatabaseHandler facilitates bidirectional flow of data that is needed to support input/output operations with clients 104, such as user requests to update agent profiles, populating the Client GUI, and sending real-time updates.

Whereas AgentServer is primarily concerned with facilitating the flow of data into the database 330, the ClientServer of an <AWSServer> object is designed to retrieve data from the database 330. Similar to AgentServer, ClientServer encrypts messages that are sent to a given client 104 and decrypts incoming sensor objects that are sent by <AWS Sensor Agent> objects through via their respective AwsServerConnections. Additionally, ClientServer supports concurrent, non-concurrent, synchronous and asynchronous, and browser-based connections.

As shown in FIG. 7, requests from ClientServer are passed to ClientRequestHandler. In general, ClientRequestHandler receives requests from ClientServer, parses such requests, and creates one of two different types of objects. One object type which may be created by ClientRequestHandler is a SQL query that inserts data such as, for example, sensor profiles, responder profiles and other profiles, into the database 330. This object type may also get the sensor data requested by the applicable client 104. The other object type created by ClientRequestHandler is a rules engine object sent to the RulesEngine.

The RulesEngine receives rules engine objects generated in response to requests from clients 104 and from AwsDatabaseHandler, and runs the rules engine 314. The RulesEngine then generates output for the requesting client 104 in the form of object data, messages, trigger profiles, etc. The RulesEngine evaluates the current conditions, comparing data with defined threshold levels and acting accordingly. An example of an exemplary user-defined rule which could be executed under the direction of the RulesEngine is set forth below:
if sensor value exceeds “x”, then send automated signal to shut down HVAC and send alerts to responders

The output produced by the RulesEngine is sent through ClientServer to the requesting client 104. In turn, the client 104 responds appropriately in view of the nature of the information produced by the RulesEngine. For example, this information may cause the client 104 to generate an alert, confirm a change to a user's profile, update a threshold level, etc. Such client responses may be in a variety of forms (e.g., email, SMS, pagers, text alerts, etc.).

### Client User Interface

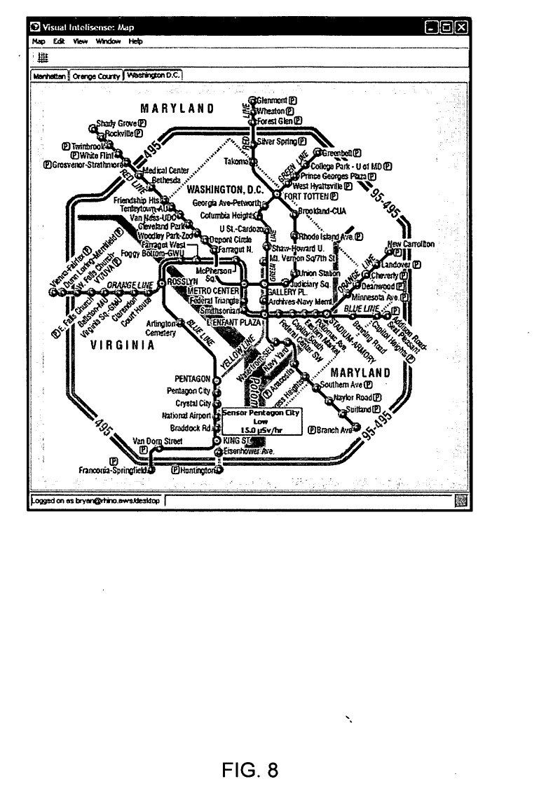

FIGS. 8-11 are screen shots of an exemplary, set of user interfaces capable of being presented by the Intelisense™ clients 104. In this regard three different types of interfaces are described with reference to FIGS. 8-11: Map Windows, Event Windows, and Administration Windows. Briefly, a Map Window is an interface within which sensor and personnel data overlay on top of regional maps; an Event Window presents the data streaming in to an Intelisense™ server 102 in a tabular fashion; and an Administration Window provides all the tools needed to manage and administer the system.

As will be described with reference to FIGS. 8-11, in the exemplary embodiment the Visual Intelisense™ Client is the main interface that agents and administrators use to interact with the system as well as to maintain it. User access to the system can be controlled at a very granular level. At the most basic level, the Visual Intelisense™ client requires a login and password in order to gain access to the system. Users can be granted privileges to individual component settings, such as specific regions, system functionality, or sensor types. In addition, different levels of authority are also provided to enable improved administration and security risk reduction; that is, users will only have access to the parts of the system that they need to access.

Map Window

Turning now to FIG. 8, there is illustrated a screen shot of a user interface of the Map Window type. The Map window is the primary visual interface for agents to use to discern the overall status of a particular region. The map information is pulled from the Visual Intelisense™ Server, so different teams accessing different such Servers may have different sets of maps to review. User interfaces in the form of Map Window may be used to display any number of different regional maps and overlaying information relating to sensors and key personnel in a visual and intuitive manner.

Event Window

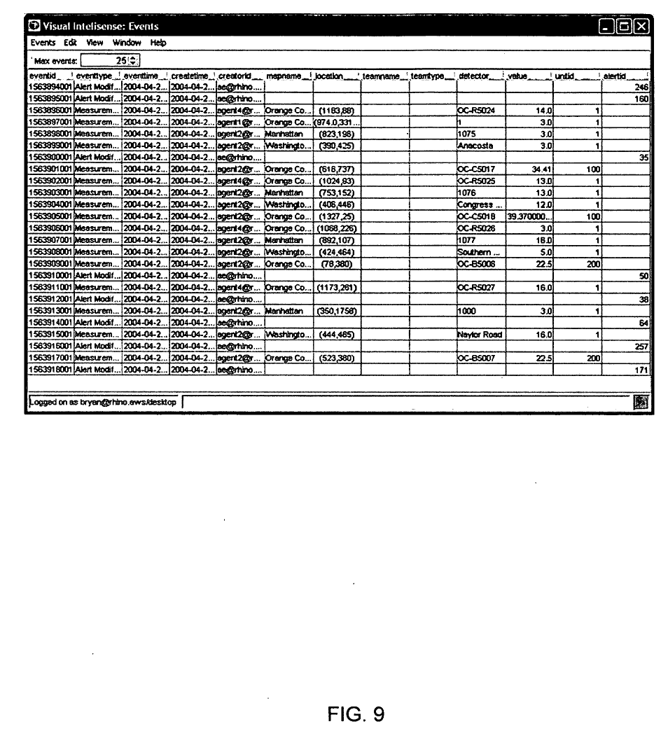

Referring to FIG. 9, shown is a screen shot of a user interface of the Event Window type. The Event Window presents sensor and responder data registered by the applicable Intelisense™ Server in a tabular format. As new data streams into the Server from remote sensors and personnel, the Server then passes the information to the Event Window displayed by the Visual Intelisense™ Client.

Administration Window

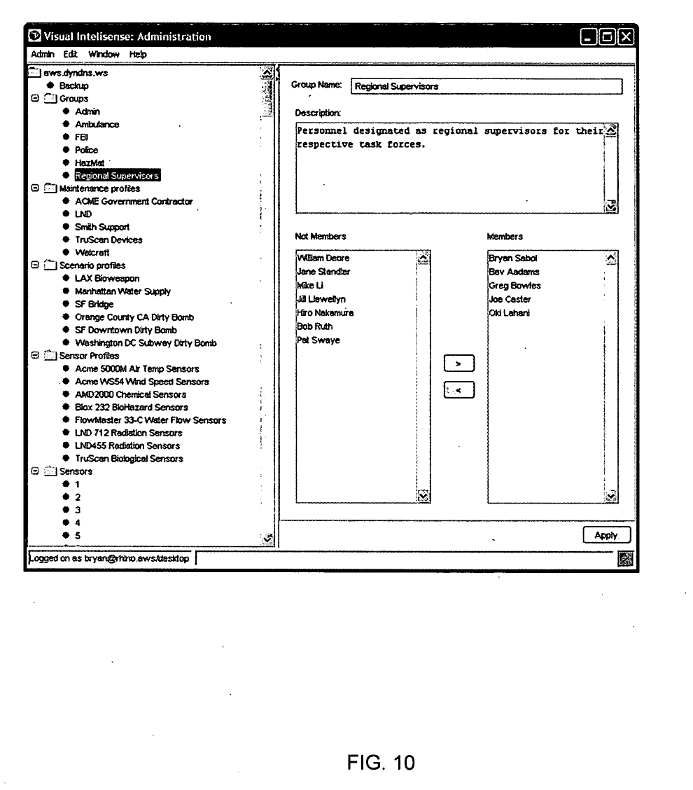

FIG. 10 depicts a screen shot of a user interface or the Administration Window type. In one embodiment the Administration Window contains all the standard administrative tools required for maintaining the system, including user management, sensor configuration, profiles, backups and archiving. That is, Users, groups, sensors, backups and archives, and profiles are all created and managed through an Administration Window. As shown in FIG. 10, the Administration window uses a hierarchical navigation tree in the left frame of the window.

Sensor Administration

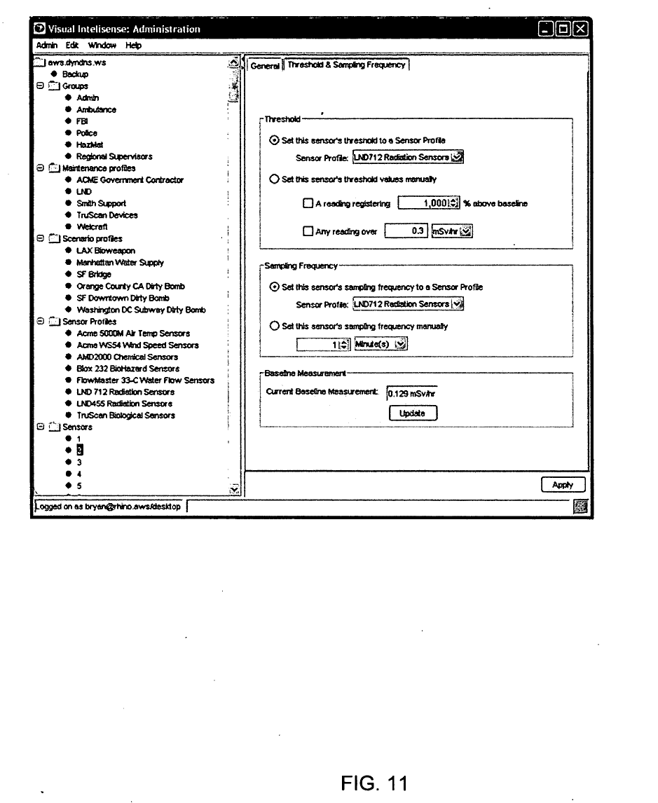

Referring now to FIG. 11, there is shown another screen shot of an Administration Window which illustrates the manner in which the settings of sensors may be adjusted. In this regard the settings of sensors may either be set individually or controlled as a group by referencing a Sensor Profile.

### Exemplary Usage Scenarios

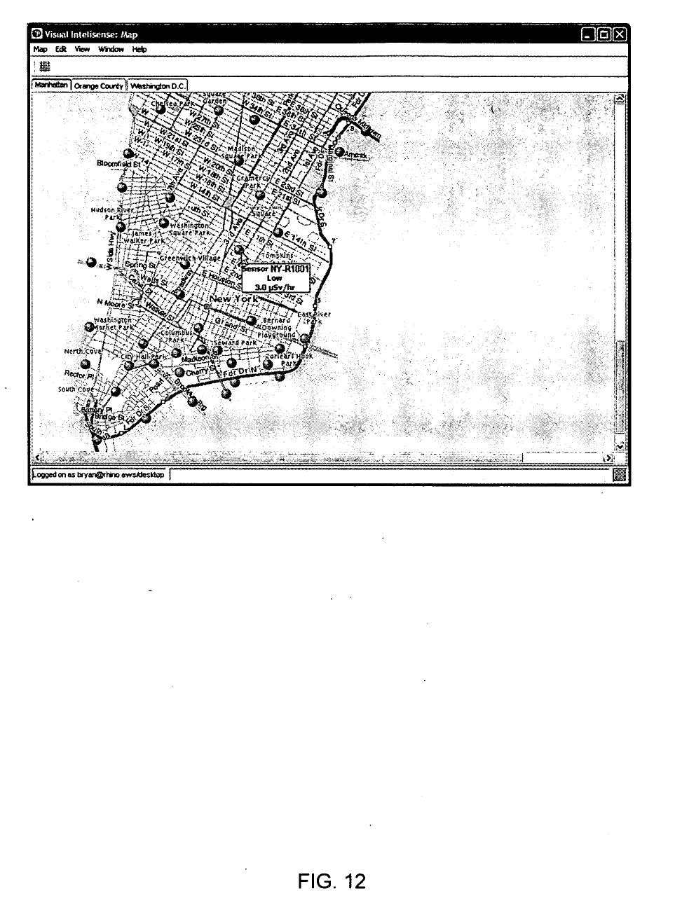

FIGS. 12-20 illustratively represent the manner in which the inventive Visual Intelisense™ system may be utilized to monitor large-scale environments, detect emergency events, alert appropriate first responders, and potentially activate automated systems.

Constant Sensor Monitoring

In exemplary implementations the Visual Intelisense™ system may operate to constantly monitor the relevant environment for signs of a terrorist attack or other hazardous event. A network of hundreds, or even thousands, of remote sensors may be harnessed, each of which streams its data into an Intelisense™ server, which then interprets their values and displays the information in the requesting Intelisense™ client.

The maps available to an Intelisense™ client are capable of displaying this potentially enormous amount of information in an intuitive, visual manner. For example, in the representation of Manhattan depicted in FIG. 12, each circle represents a radiation sensor that is feeding a constant stream of data to the Intelisense™ server. Since all the circular representations of sensors overlaying the map of Manhattan are green, it is readily apparent to an informed user that all therefore indicate normal (low) radiation levels. Depending on the installation's requirements, other sensor types including chemical and biological agent detectors, wind speed and direction, air temperature, water flow, etc, could be displayed. In the exemplary embodiment it is thus possible to review an entire sensor set's overall status (e.g., green) at a glance, as well as to obtain additional by positioning a user interface pointing device (e.g., a mouse) over a sensor icon of interest.

Instant Alerting of a Detected Event

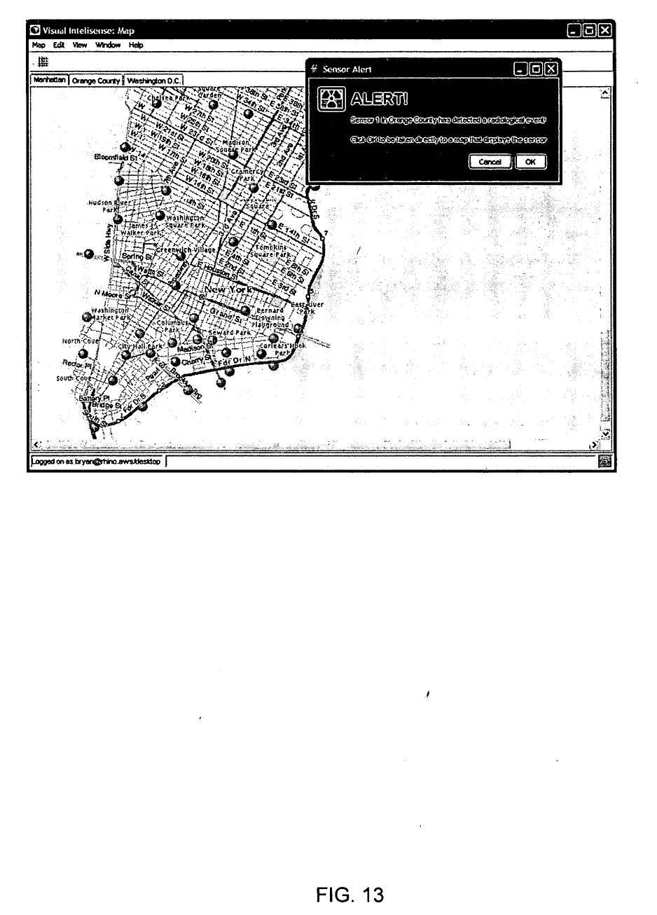

Once a threshold event is detected, Visual Intelisense™ can automatically activate emergency response systems, forwarding relevant information concerning the nature of the attack. As shown in FIG. 13, as soon as a sensor sends a reading of sufficiently elevated radiation (a “threshold event”), Visual Intelisense™ instantly sets off an alert. Regardless of the location of the detected event or the time of day, the system immediately jumps to the map that contains the alert sensor and centers the alert sensor in the middle of the screen.

Real-Time Situational Awareness

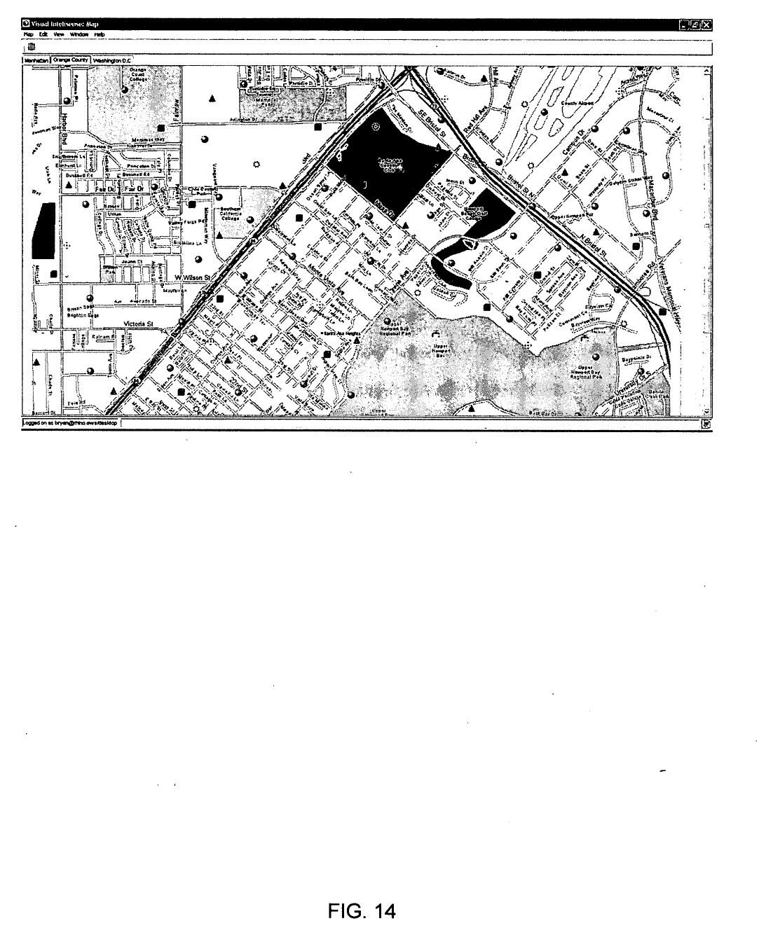

Turning now to FIG. 14, an illustrative representation is provided of the spread of radiological contamination arising from detonation of a radiological device proximate the regional airport in Orange County, Calif. The spread of the radiological contaminate is clearly indicated as green “normal” status sensors turn orange, then red as the radiation spreads (by climatic conditions, people, cars, etc.), and the level of contamination increases. Although in an exemplary implementation seven levels of intensity are supported over a predefined color spectrum, the number of levels can easily be customized in accordance with specific installations.

FIG. 14 also indicates that a variety of different types of sensors may be deployed. In addition to the radiation sensors (spheres), the installation represented by FIG. 14 includes biological sensors (cones), chemical sensors (cubes), wind speed and direction (compass), water flow (faucet), etc.

Enabling Response Through Interoperability

To help coordinate first responders and improve their response time to an emergency event, Visual Intelisense™ provides Scenario Profiles, which let users specify which entities and personnel should be notified and what automatic response systems should be activated due to a particular event.

Depending on how the Scenario Profile is configured, the Intelisense™ system may either instantly respond or wait for authorization before proceeding. It may also be specified that alerts be generated based upon more than a single sensor prior to initiation activation processes in order to reduce the chance of the Intelisense™ system responding to a malfunction.

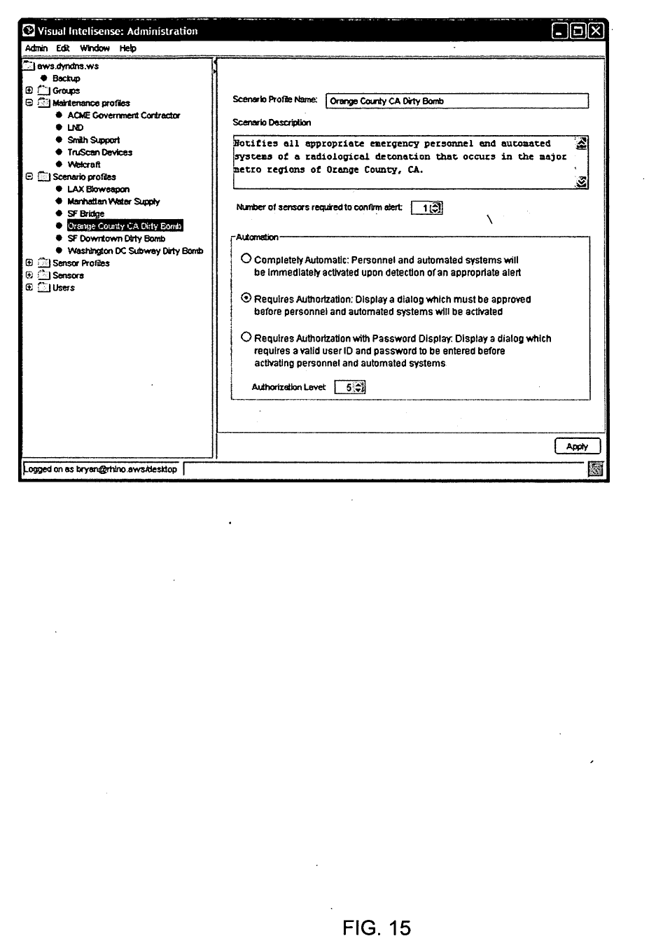

Referring to FIG. 15, an exemplary user interface screen shot is depicted of a Scenario Profile enabling specification of (i) the entities or individuals to be notified when a particular event occurs, (ii) the level of automation of the Profile, and (iii) the number of sensors that should detect an event before activating the Scenario. In the specific case of FIG. 15, it has been specified that an alert is to be raised when one or more sensors in the monitored area register threshold levels of radiation.

Automated Personnel Notification and System Activation

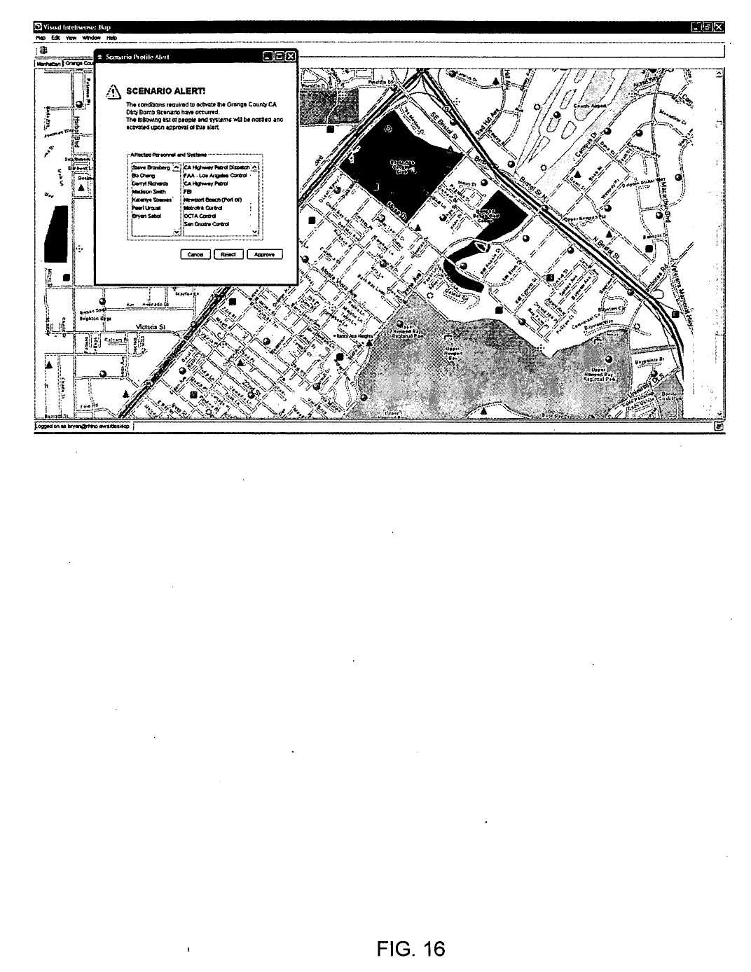

96 FIG. 16 illustrates the manner in which the Scenario Profiles of Visual Intelisense™ allow instant notification of the appropriate set of first responders and activation of emergency response teams for any defined event. In the exemplary embodiment once the system determines that an event matches the activation requirements of a Scenario Profile, it either instantly activates or waits for approval. In the specific example of FIG. 16, an alert displays all the key personnel who will be notified and systems that will be activated when the Approve button is clicked. Once activated, a Scenario Profile notifies the entire set of key personnel, sending alerts to pre-defined email clients, pagers, cell phone text messages, wireless PDAs, blackberry devices, and virtually any other type of communication device.

In the exemplary embodiment as many different Scenario Profiles can be defined as are needed. Each profile specifies a particular combination of event, geographic location, and set of notified first responders and activated emergency response systems. Once defined, the profiles are ready to be activated the moment the appropriate criteria are met.

Similarly, the Scenario Profile can also send the appropriate activation codes to existing emergency response systems. This capability of activating a pre-determined set of response systems en masse is another key factor in improving response time.

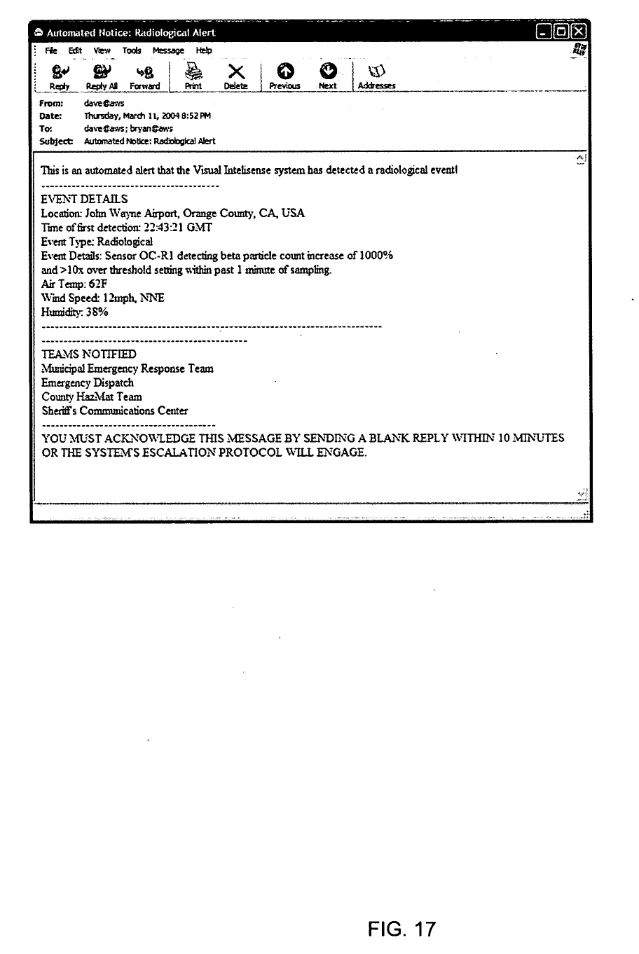

FIG. 17 depicts a sample email message that may be automatically sent to a first responder. Although there are a variety of notification mechanisms that can be used, email supports a larger amount of text than pagers or cell phone text messages. First responders who receive their alerts via email can therefore review the nature of the event and see critical details such as the event type, its location, weather conditions, and other key personnel and systems that have been activated.

Exemplary system implementations may also offer a workflow engine that allows a user to specify. escalation procedures. As noted in the last sentence of the email shown in FIG. 17, the alerted person should respond within a certain period of time or alternate emergency personnel will be contacted. This escalation capability is a key factor in ensuring that the appropriate parties are not only notified, but respond in a determined period of time.

Visualizing the Scope of the Emergency

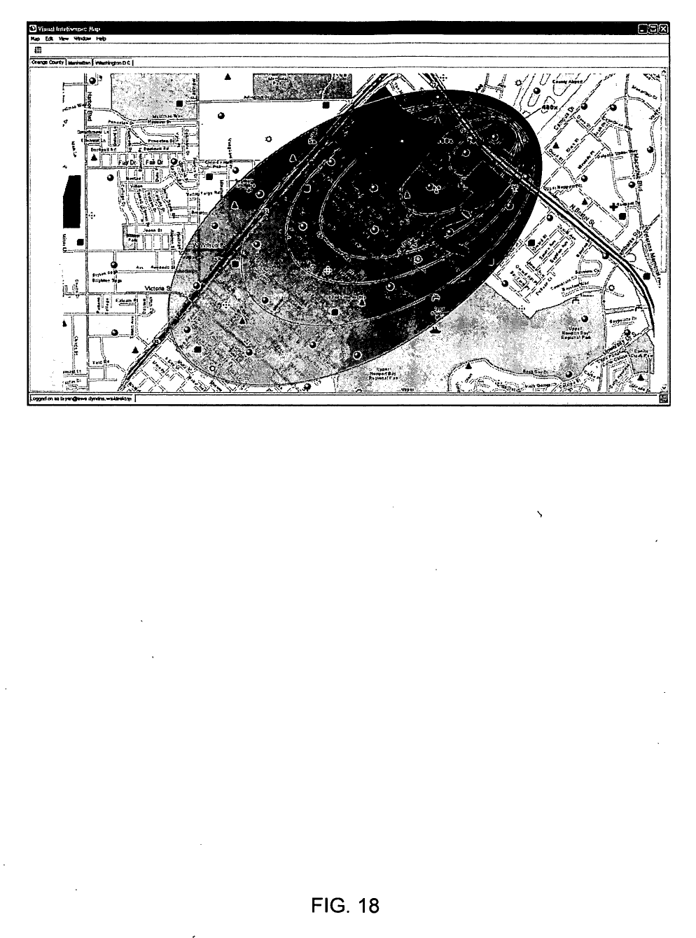

FIG. 18 is a user interface screen shot illustrating the manner in which Visual Intelisense™ is capable of displaying overlays to assist in visualization of the nature and spread of a potentially hazardous event. In the case of FIG. 18, the representation indicates that radiation levels very substantially above background levels exist at the source of the detonation, with additional sensors showing the movement and drop-off of radiation as a function of distance and wind currents. In this regard FIG. 18 is representative of the various types of overlay tools (e.g., gradients and plumes) which may be combined to assist in visualizing the impact of an event and in tracking its spread.

Overlay of Critical Data

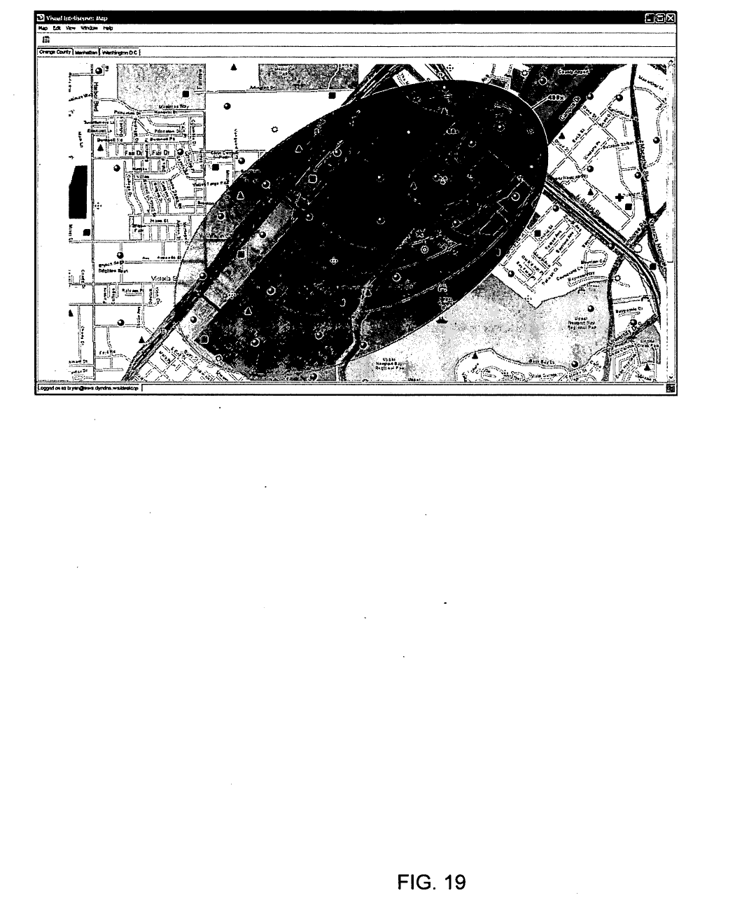

FIG. 19 is a user interface screen shot demonstrating the ability of Visual Intelisense™ to overlay key economic data along with information concerning the emergency or hazardous event being monitored. The type of economic-related information which may be represented by such overlays may include, for example, key transportation routes, warehouses, and distribution centers for manufactured goods; financial institutions such as banking centers, stock exchange facilities, and brokerage houses; and food processing centers, like supermarkets, open-air farmers' markets, wholesalers, canneries, marinas, etc. In the specific case of FIG. 19, the blue areas represent key transportation routes, facilities, and distribution centers; the green areas represent high-density housing, and yellow areas indicate key government facilities including hospitals, police, courthouses, etc. In this way FIG. 19 demonstrates that, through use and integration with existing mapping (GIS) technologies, a variety of commercial data can be overlaid with the event's details in order to generate specialized maps. It is a feature of Visual Intelisense™ that these maps may provide unique insights into the economic impact an event may have on businesses. The ability to juxtapose the spread of the radiation, chemical, etc., with other types of physical data provides a unique view of the situation and will help authorities make the right decision for deployment of limited resources.

Real-Time Tracking of First Responders

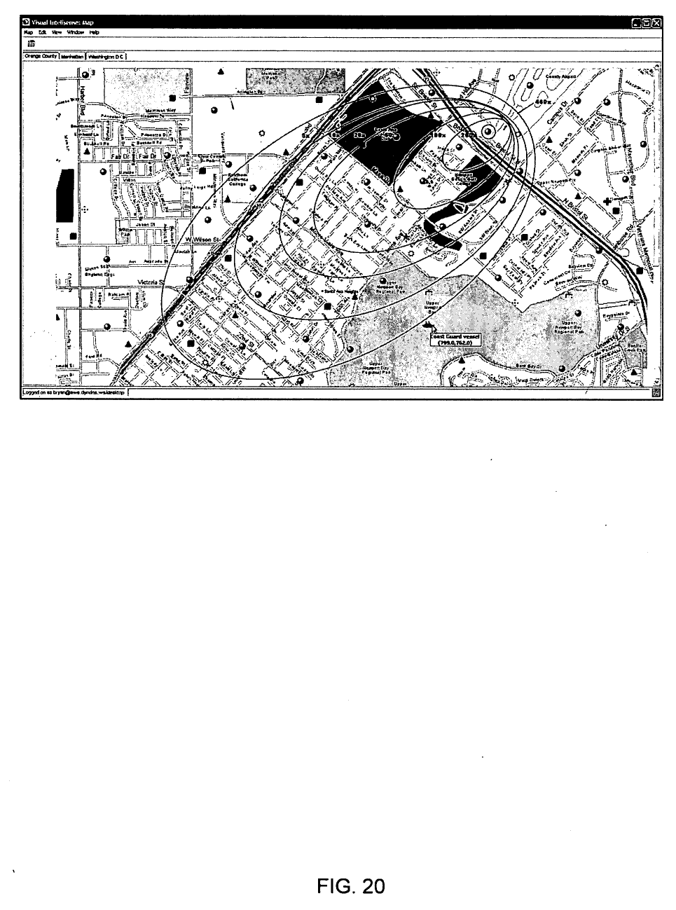

FIG. 20 is an exemplary user interface screen shot representative of one manner in which emergency personnel equipped with portable positioning indicators may be tracked on a given map. In particular, through the use of handheld, vehicle-based, and other mobile positioning devices that interface with the Intelisense™ server, multiple response teams are uniquely identified and tracked. This enables icons of the type present in FIG. 20 to be overlaid on a given map in order to provide a visual representation of the location of such response teams relative to areas of interest.

By interfacing with existing tracking systems, Visual Intelisense™ can visually identify and display each team member and track their position and movements in real-time on the event map. GPS technology can also be leveraged to provide portable teams, detection units, and others the ability to be tracked anywhere in the world. This permits-Emergency commanders to view at a glance the location and status of each team that is present at an event site. Moreover, commanders may initiate the sending of messages to such teams directly from the user interface in order to, for example, coordinate their actions.

### Scale of Visualization; Layer Filtering; Historical Data Representation

A number of aspects of the Visual Intelisense™ platform render it particularly suitable for visualization and analysis of emergency events. In this section three of these differentiating features of the platform are described; namely, scale of visualization, layer filtering and historical data representation.

Scale of Visualization

The Visual Intelisense™ platform is capable of accommodating visualization and management of a broad range of scales of geographic locations. Visualization on a macro scale is handled in two ways. The first is to simply display an all encompassing map, with information intelligently filtered so as to not overwhelm a user, but still present critical information in a timely and easy to understand fashion.

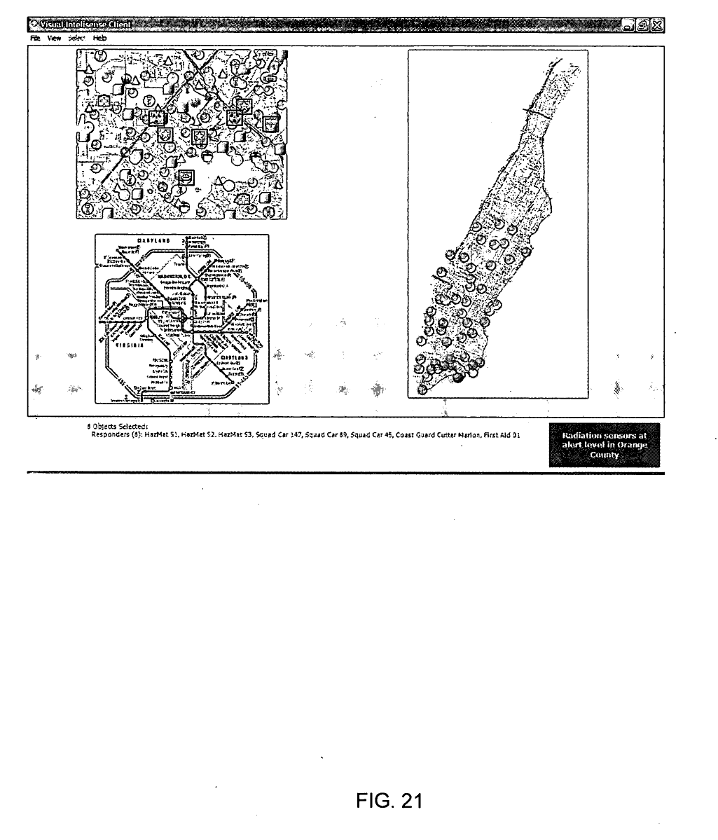

FIG. 21 provides an illustration of the results of the second method, which comprises dividing the large scale map into multiple, smaller maps which are tiled to fit the display of the Intelisense™ Client. In the example depicted by FIG. 21, three separate locations are tiled in a manner capable of fitting within the browser window of a standard laptop display. A user with a custom wall or equivalent display could tile countless maps for a complete view of critical locations. This particular ability of “tiling” multiple maps on the same screen can also be useful for monitoring the same location from different angles (e.g., use an elevation map in conjunction with traditional “bird's eye” map views).

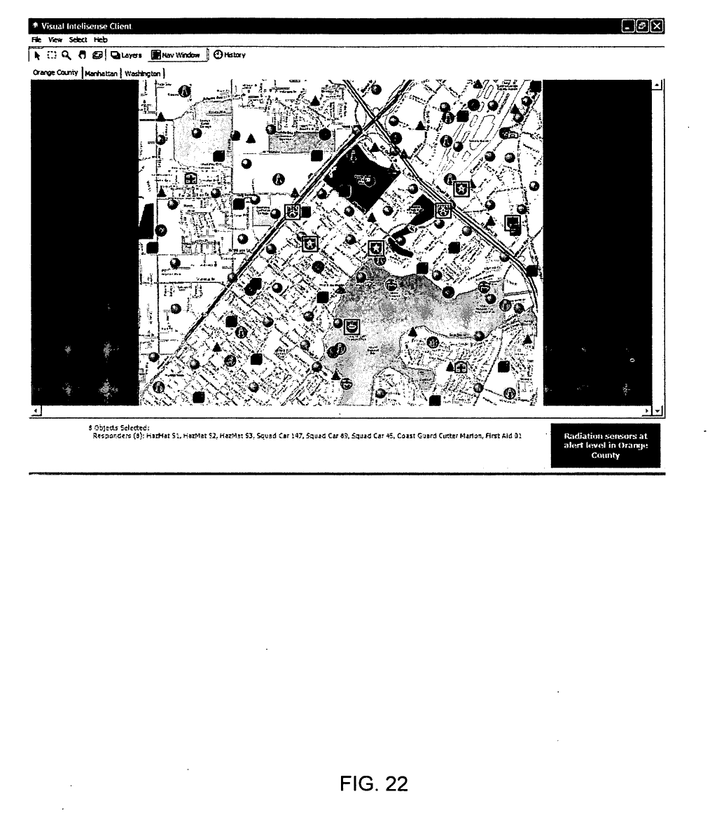

Turning to FIG. 22, the next level of scale is an intermediate view, displaying a single, entire location, such as a city or county, fully within the browser window. Multiple locations can be placed in tabs for quick navigation between them. On this scale more detail is shown, and more visualization options are presented.

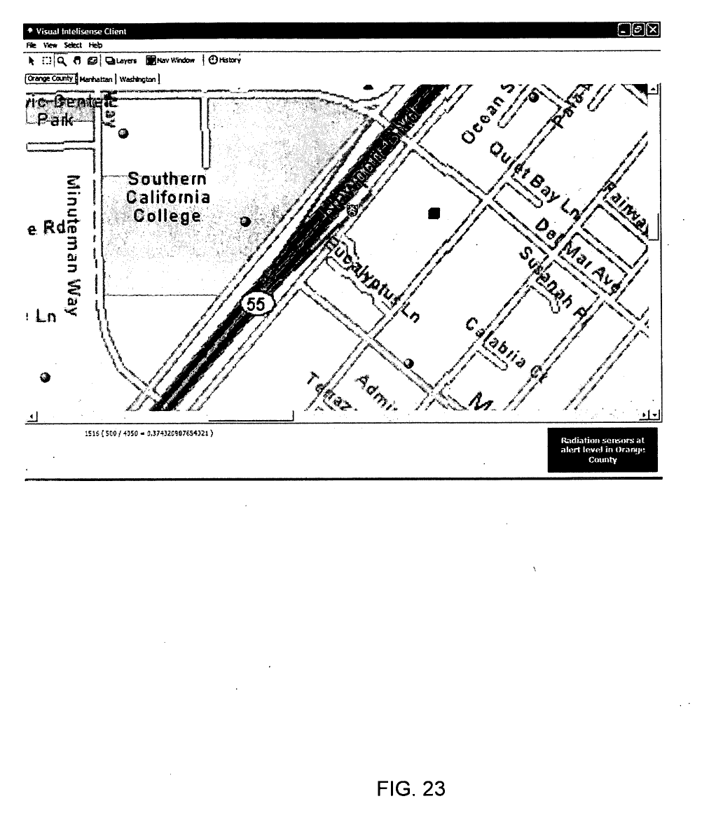

Finally, FIG. 23 illustrates the final level of scale, which is capable of being achieved using various “zooming” functions provided through the user interface. In this case the user is permitted to zoom into a location indefinitely, limited only by the resolution of the underlying map.

Layers Description

All items that can be displayed on the map, such as sensors and responders, as well as any graphical overlays, such as economic information or radiation plume predictions, can be filtered in and out of view using a layers dialog.

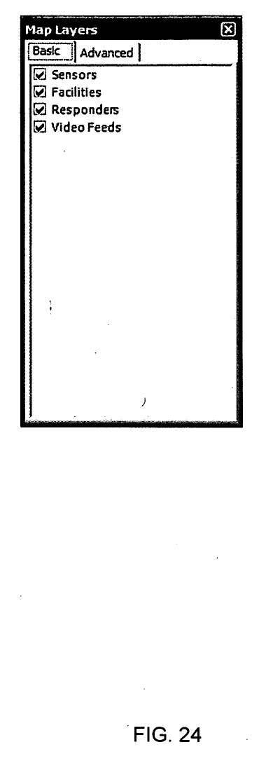

FIG. 24 depicts a screen shot of an exemplary Layers dialog, which is divided in two tabs. Selection of the “Basic” tab results in display of a Basic view. The Basic view shows only the highest elements of the object architecture and allows the user a general level of control over what is displayed on the map.

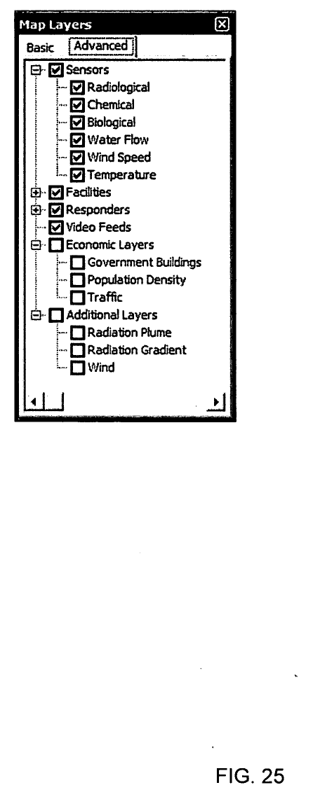

FIG. 25 is a screen shot illustrating the Advanced view of the Layers dialog, which is invoked by selecting the “Advanced” tab. The Advanced view presents more advanced options and more finite control over the layers to display. The layer checkboxes are displayed in a classic tree view. Toggling the checkboxes for a top level node draws or hides all sub-levels of a type of object. Using this control, the user can drag items up or down in the list to change the top down order of the layers drawn on the map (i.e. change which layer is drawn on top of another).

Historical Data

One differentiating feature of the Visual Intelisense™ platform relates to its ability to aggregate and display historical information. For example, in the exemplary embodiment the Visual Intelisense™ server maintains a historical database of every reading from each MapObject (unless otherwise specified by the user) which can be queried using SQL to develop powerful visual representations, tools, and datasets for many possible applications. The user is unaware of the use of SQL, as the user interface of the Visual Intelisense™ client provides the means for choosing time spans, layers, and objects to be included (SQL queries can be entered directly by more experienced users). As an example, in the case of detonation of a radioactive device, a query could be submitted in order to request display of the last 20 minutes of data for all radiation sensors, and emergency response teams (e.g., “hazmat” teams), and police officers. The query could, for example, also request overlay upon the display of a representation of the radiation plume resulting from the detonation.

Exemplary Uses of Historical Data

A first use of historical data is to export the data for evaluation using external software (e.g. GIS). This exported data could be evaluated for virtually any purpose that a user could conceive, such as analyzing responses to an emergency, developing better models for analyzing radiation plume dispersion, or analyzing population movement data.

A second key use of historical data is instant evaluation of recent history during an emergency or other important event. Recent history can be quickly downloaded and viewed to evaluate movement of responders and developments in event status. For example, this can be used to determine the duration and intensity of exposure to radiation a responder may have incurred.

A third usage of historical data is to train responders, software operators, or incident commanders by studying footage of prior events. Time frames including key events can be downloaded, saved, and later replayed any number of times for demonstration and instructional purposes.

Visualization of Historical Data

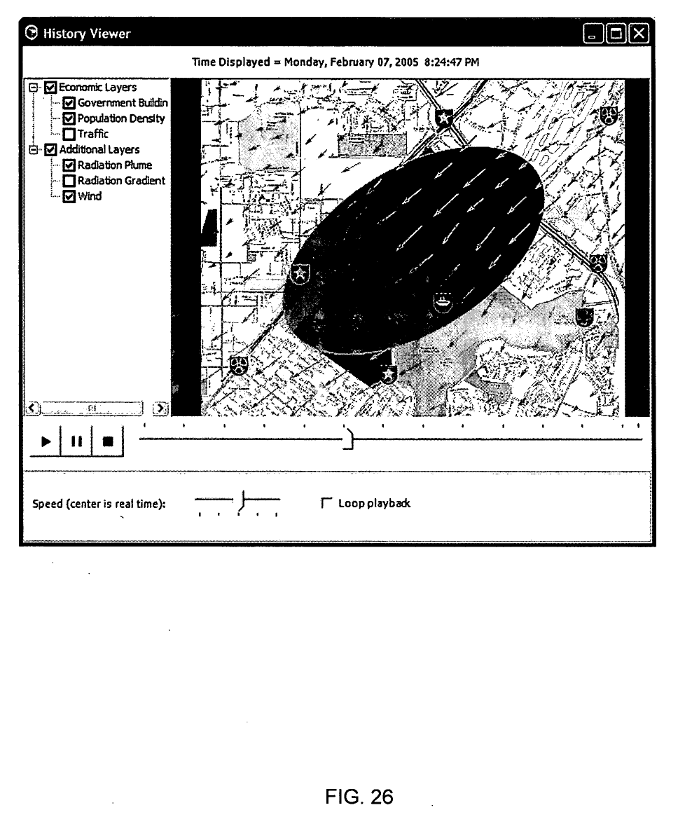

FIG. 26 is a screen shot of a History Viewer interface which illustrates a first mode of historical data visualization. Consistent with this first mode, historical data is presented using an animation-style display of events. In addition to standard methods of controlling the animation, the window gives the user control over what data or layers are displayed, in a fashion identical to the layers window for the main application.

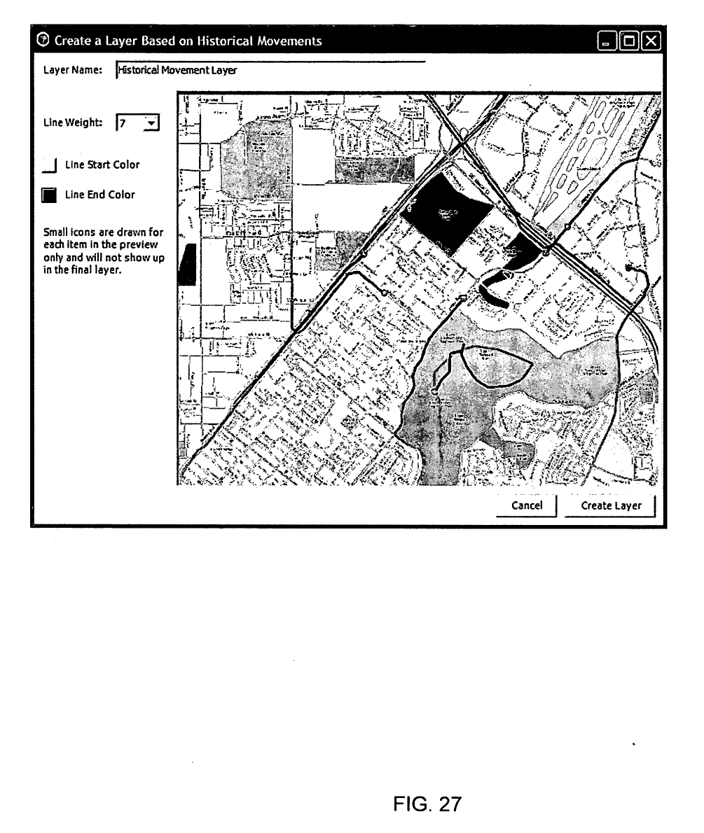

Referring to FIG. 27, there is shown a screen shot of a user interface which illustrates a second mode of historical data visualization. This second mode of visualizing historical data involves creating static graphical representations of various aspects of the data, which are then overlaid upon the underlying primary map view. For example, in FIG. 27 a path that an object or group of objects has followed over the course of a given time span is depicted in an overlay placed over the underlying primary map view. When a user has created graphics layers using this method, they appear in the layers window and their display within the viewing window can be selectively toggled on and off like any other layer.

### Visual Intelisense™ Exemplary Software Architecture

This section describes the software structure and other architectural attributes of an exemplary embodiment of the Visual Intelisense™ system of the present invention.

Introduction

As discussed above, in one embodiment Visual Intelisense (“VI”) comprises a software application designed to run on top of sensor networks and existing control systems. A facility's existing sensors, along with improved sensors developed in the future, can be integrated with Visual Intelisense and placed in substantially constant communicating with the Intelisense Server. Visual Intelisense converts the data streaming from a potentially vast sensor network into the real-time, visual and actionable intelligence that is needed before, during and after an emergency event.

In the exemplary embodiment Visual Intelisense constantly and automatically analyzes sensor readings for the detection of events having environmental consequences such as, for example, industrial accidents and deliberate terrorist attacks. Once an event occurs, alerts may be automatically sent to the proper authorities, emergency operations centers, and healthcare facilities.

During an emergency event, Visual Intelisense gives responders the ability to “see” and understand the “big picture” in only a glance. All of the information critical for response teams to make fast decisions is overlaid on a map of the affected area. As the event unfolds, Visual Intelisense automatically changes the “big picture” in real-time, providing the true situation awareness that responders need to establish command and control of the situation.

Visual Intelisense also leverages its intelligent network to constantly monitor and report on sensor health. Maintenance profiles are used to detect error conditions from sensors, which Virtual Intelisense uses to automatically forward diagnostic details to the appropriate vendor and/or maintenance contractor.

System Overview

Exemplary implementations of Visual Intelisense are composed of a set of sensors, servers, databases, and clients that monitor a geographic area for events and display their status.

The Intelisense Server is a back-end component that receives, processes, stores, and forwards the messages from the sensors.

The Intelisense Client is the user application that interfaces with the Server. The Client provides notification and status displays, as well as administration and configuration functionality. These tasks should also be available via a command line or scripting interface. Sensors are devices that provide a measurement of interest for a known location (either fixed or mobile via GPS) and pass their information through the sensor network to the Server. Sensors are composed of the following sub-components:
A detector which is a hardware device that makes the actual measurement.
A messaging processor and network device that allows the sensor to send a message using TCP/IP.

A Sensor Proxy is a computer that handles the messaging and interfaces to hardware detectors that use a simpler protocol most often via a serial communications port. A Sensor Proxy can attach hundreds of detectors to the system in this way.

Services are the software processes that run on host computers. A host computer can have one or more services running on it.

A database is a server that stores the sensor measurements, configuration, and other data. There may also a GIS database that contains the maps and/or other vector data which is leveraged by the system, but shouldn't be considered part of the system itself.

Target User Profiles

Primary Users

Primary users are defined as those people who will spend a significant amount of time working with the technology.
Facility Security Personnel: The Intelisense Server will typically be installed in the Security office or other secure IT room on-site. Personnel whose main responsibility is the regular monitoring of the facility (be it a government building, a refinery, resort, civic center, etc.) will use the Intelisense Client on a regular basis to monitor the environment, manage access, define profiles, etc. Security officials will also be the primary recipients of the system alerts that occur when an emergency is detected. Security personnel will therefore use the bulk of the Client interface, expect possibly the administrative and maintenance areas.
   Secondary Users

Secondary users are those who interact with the system on an infrequent basis, typically for administrative tasks, or to access logs for use in pattern analysis.
IT Managers: Will perform the appropriate administrative tasks to maintain the system. IT managers or others with appropriate authority will access the administrative screens to define backup locations, add users, change passwords, and assist in defining various profiles when necessary.
Security Supervisors: Security supervisors may come into contact with VI typically only to create and/or modify user accounts. They may also be involved by being in the “chain of command,” meaning they could be a designated contact (or an escalation contact) in a scenario profile who would be automatically alerted when the system detected an emergency.
Pattern Analysts: Because VI logs all the sensor data it receives, these raw data points can be ported to other systems on a semi-regular basis to search for large-scale patterns.
   Tertiary Users

Tertiary users are only peripherally involved with the system. Most commonly, these users are simply recipients of an alert that was generated by the VI system. They may not even be aware that the information they are receiving originated from VI. These users include:
EOC Members: Could receive alerts through their designated communications channel(s) if they were designated contacts for a pre-defined scenario profile. If desired, the EOC facility could also maintain one or multiple copies of the Intelisense Client that they could use to monitor the emergency in real time.
Incident Commanders: Same usage as EOC members above.
Plant Managers/Directors: Same usage as EOC members above.
Maintenance Personnel: Since VI has the capacity to monitor its own health, the system could detect a malfunctioning or unresponsive sensor in the network. By associating each sensor with a maintenance profile, the system could then automatically send details of the malfunction to the company's maintenance contractor or other party as defined in an SLA.
   Major System Features

This section provides details about each major system feature.

Collect and Analyze Sensor Data

Description

The most fundamental capability of the Intelisense Server is to collect and analyze data from a sensor network.

Stimulus/Response Sequences

1. Server will constantly receive sensor data streaming in over the sensor network.

2. As each new data point is processed, its value is pushed from the Server to all active Clients.

3. If any particular sensor's value reaches a threshold level as specified in the Sensor Configuration dialog (see Remote Sensor Configuration below), then a System Alert is generated, which should be displayed on all active Clients.

4. If the emergency event unfolds to a level that meets the criteria defined in a Scenario Profile, then a Scenario Profile Alert is generated, which is displayed on all active Clients.

Functional Specifications

SPEC-1: The Server should be able to use its rules engine to determine whether a particular value from a specific sensor matches a pre-defined rule to activate an alert.

SPEC-2: “Objects of interest”—not just sensors, but responders, and other data types should be dynamically recognized by the server as soon as valid messages from the “object” are sent to the Server. The Server should be able to recognize the type of object and plot its location and details on the appropriate regional map.

Remote Sensor Configuration

Description

The system should be able to remotely configure each sensor in the network. The interface and list of desired configuration parameters are listed in this section; the required hardware interface and 2-way communication channel needed for the Server to propagate configuration changes out to the sensors are described below with reference to the Detector-Mote Interface.

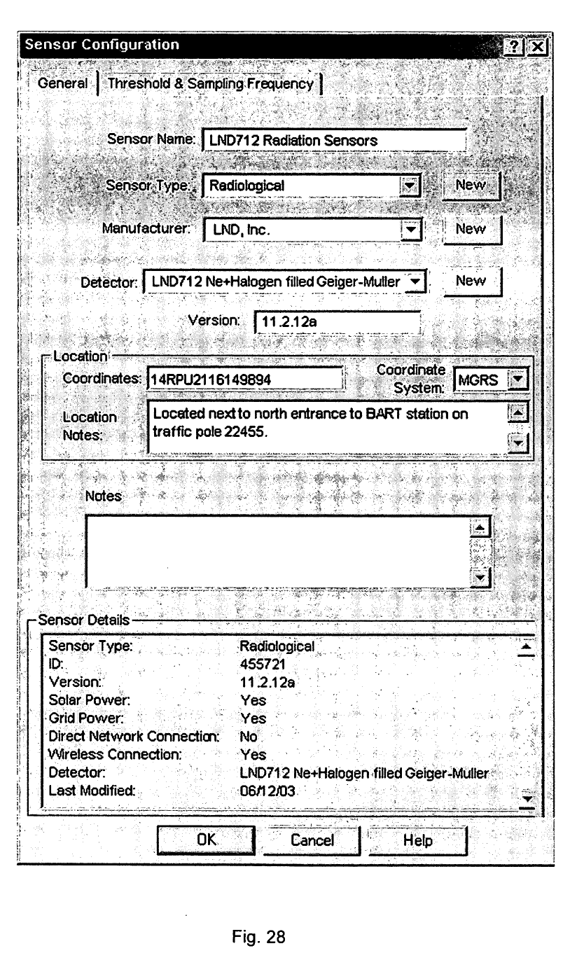

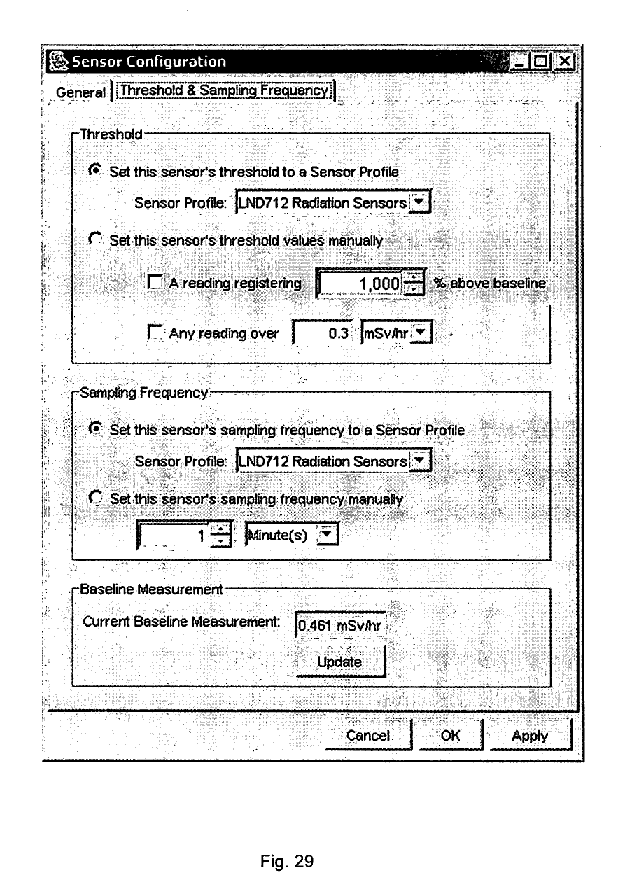

Stimulus/Response Sequences
The Client application will provide a configuration dialog, which will be the primary mechanism by which users can configure each sensor.
Access to this dialog should be available at a minimum by clicking on an object on the map screen which represents a specific sensor. Other mechanisms should include selecting the appropriate sensor from the List View, or possibly selecting a sensor from a menu/submenu hierarchy.
Screen shots of an exemplary GUI are depicted FIGS. 28-29. Each area within the GUI is described in the following Functional Specifications.
   Functional Specifications

SPEC-1: Sensor Name: Users should be able to name the sensor, independent of the unique ID the system allocates for it.

SPEC-2: Sensor Type: There should be a mechanism to specify the type of sensor, along with an edit function to add or delete to/from the type list.

SPEC-3: Manufacturer: This field would probably be better served being titled “Maintenance Provider,” as the idea is to provide a reference to a company POC who has an SLA with the client company. If/when a malfunction is detected in this particular sensor, this field would provide a lookup to the proper contact info. The Server would then email the contact, notifying them of the need to repair/replace, providing specific location information, as well as log details that describe the nature of the malfunction. There should also be an edit function to allow users to add new contractors and modify and/or delete existing ones.

SPEC-4: Detector: There should be a field where the user can identify the specific type of detector that is contained in the sensor package. This information needs to be granular enough to identify brand name and model. As with other fields, this one requires an edit function to define new detectors as well as edit/delete ones no longer used.

SPEC-5: Version: The editable version field is to describe a particular version of a given detector model.

SPEC-6: Location: This is editable for fixed sensors, but should be read-only for mobile agents. Sensors also can optionally belong to a Region Profile, whose inclusion would be determined by this location data.

SPEC-7: Notes: This editable field lets the user provide any additional information that is relevant to the sensor. In particular, the location of the device, date last serviced, whether it's near a naturally high radiation source, etc.

SPEC-8: Threshold (group): Each sensor, regardless of its type, should have a threshold level. It is this value that is used to identify normal conditions versus unusual conditions versus emergencies. Thresholds values and types will depend on the type of sensor (e.g., a wind sensor may have a wind speed threshold, but a radiation detector as shown in FIGS. 28-29 may have both a % increase in ambient radiation, plus an absolute level of radiation threshold). The system should also help to simply the administrative overhead that could be required to configure-then update-hundreds or thousands of individual sensors. Therefore, each sensor should have the option of having its threshold settings either controlled directly within this configuration dialog, or to be controlled instead by a Sensor Profile. If the user decides to configure this sensor via a profile, they should be able to select the existing set of available (and proper) profiles from a list. Otherwise, the user should be able to manually enter both percent over baseline values as well as absolute reading levels into the dialog.

SPEC-9: Sampling Frequency (group): Another critical parameter for all sensors is the sampling frequency, which determines how frequently the sensor should record its ambient values. As with the Threshold group, sampling frequency should also be controlled by a Sensor Profile, as well as being manually set within the dialog.

SPEC-10: Reporting Frequency: This concept is similar to the Sampling Frequency, but it controls how frequently the sensor transmits its collected data. By definition, the reporting frequency can be no shorter a time period than the sampling frequency.

SPEC-11: Baseline Measurement: A baseline measurement is a moving average of the regularly sampled values. Thus, it provides a good indicator for what is considered “normal” for a given area. Although it may not be applicable for all detector types, a good example of its usage is for radiation detectors: some areas may have a naturally higher background radiation level; their baseline measurement would then be higher than other areas. By comparing the delta—the rate of change of the background values—users may want to set a special alert to warn of a cumulative effect. In terms of functionality, at minimum this value should not be editable, but just show whatever the current MA is. The user should be able to reset the sampling rate, however. Additional functionality could be to provide advanced features that let you manipulate the moving average (length of average, make it weighted, exponential, etc.).

SPEC-12: Sensor Details: Read-only information concerning the sensor. The particulars depend on what information is/can be broadcast from the sensor, plus what settings data is appropriate to list. Some items, such as detector type, don't seem to apply as we have the drop-down controls for those values.

Map Views and Icons

Description

The Map View is the primary visual interface that users access in the Client to be able to gain situation awareness—both for ongoing security monitoring as well as for emergency response—in a given geographic area.

The interface and list of map-specific functionality are listed in this section. The overlay functionality that works in tandem with the map view is described below with reference to the Map Overlays Support.

Stimulus/Response Sequences

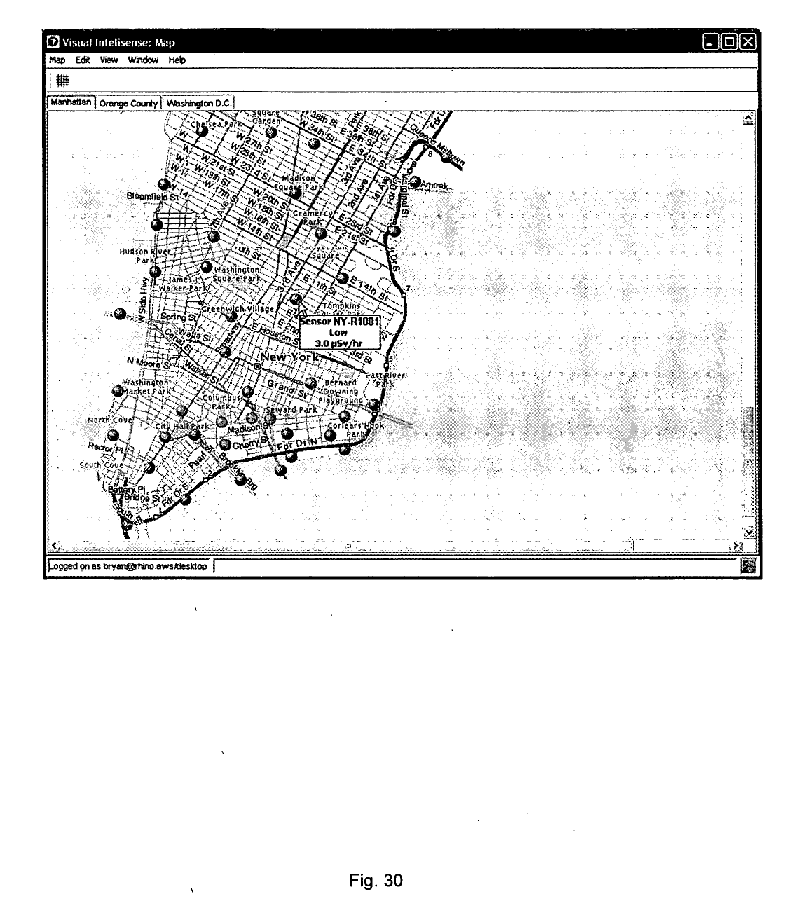

The Client application will provide a window (or windows) to display one or more maps.
A screen shot of an exemplary simple GUI is depicted in FIG. 30. Each area within the GUI is described in the following Functional Specifications.
   Functional Specifications

SPEC-1: The system should be able to display as many maps as are appropriate for a particular installation.

SPEC-2: Each map should be scalable, providing a zoom capability that not only adjusts viewing scale, but also re-draws all appropriate objects that are identified within the viewing area.

SPEC-3 There should be an easy way to navigate within and between maps, be it with tabs, a “miniature map” that shows a rectangle for the selected area within a larger space, etc.

SPEC-4 Each map window should have a toolbar to access various functionality, including overlays, scale, measurements (e.g., US vs. metric), etc.

SPEC-5 These maps should be “served” from some storage location.

SPEC-6: Should display icons for sensors. Sensors display as spheres, and are green for normal condition, red for alert condition, or gray for malfunctioning/offline state.

SPEC-7: Rolling the mouse over a sensor icon should cause a popup window to appear with the object's ID, name, condition (normal, alert, offline) description, and most recent measurements. Double-clicking a sensor icon should open the sensor's configuration dialog.

Map Overlays Support

Description

While the map itself is important to help users visualize a certain space, in exemplary implementations the bulk of the detail of the information presented (e.g., location and status of sensors; personnel tracking; meteorological information, economic data, etc.) will typically be in overlays.

Stimulus/Response Sequences

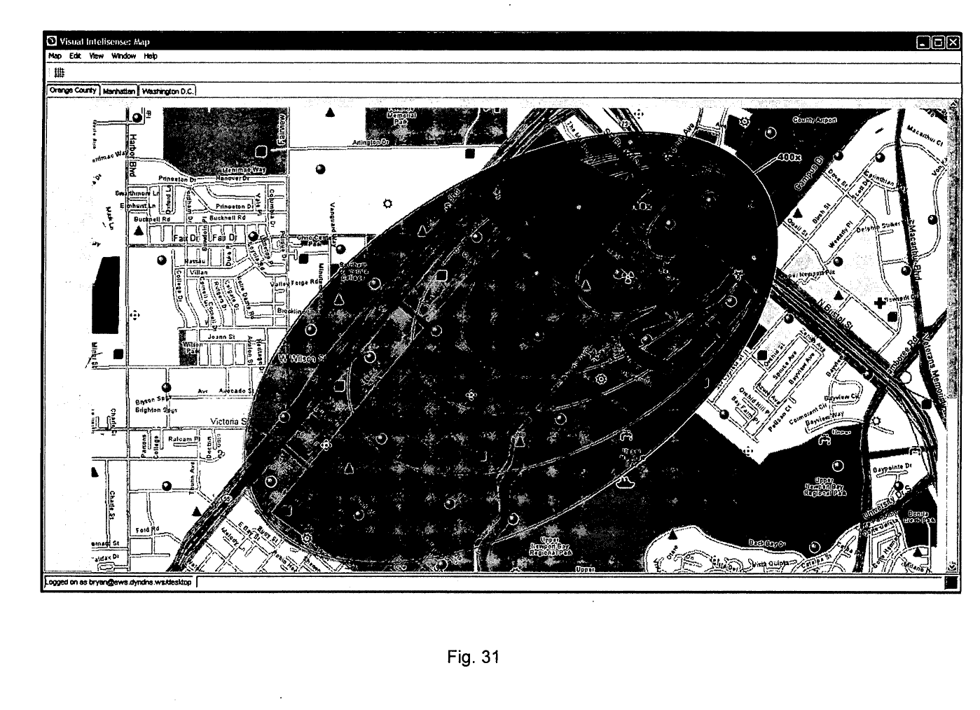

Turning to FIG. 31, there is shown an exemplary map window. The map window as described in the previous section forms the basis for the user experience for any combination of overlays. Depending on the data available, the user will choose which set of overlays to activate via the Overlay Toolbar. The overlay for the sensors will be active by default.

Functional Specifications

SPEC-1: Each map window should support any combination of independent visual overlays.

SPEC-2: Each overlay should have its own control button in the Overlay (or other) Toolbar to toggle it on and off.

SPEC-3: Should provide a sensor overlay that displays all sensors related to that map/area (this overlay is active by default)

SPEC-4: Should provide a coordinate overlay that displays a grid with X,Y cords

SPEC-5: Should provide a gradient overlay that displays a set of circles/ovals with adjustable levels per isobar, etc.

SPEC-6: Should provide a concentration cloud, or “plume” overlaying the affected area. The levels over the threshold displayed should be adjustable (i.e., plume displays any concentration over threshold level; or plume represents concentrations from 2× to 500× threshold levels).

SPEC-7: Provide a probability map (e.g., Gaussian) that could be used to estimate where the near-term movement of materials would go due to weather and/or other input conditions.

SPEC-8: Provide a set of economic overlays; each separate item providing different data like major transportation arteries; hospitals, police, and other emergency facility locations; dense population areas, etc.

SPEC-9: This overlay would place arrows indicating strength and direction of wind currents. Adjustable options include the minimum strength of wind to be mapped, etc.

List View

Description

The List view is an alternate means of reviewing data. Rather than displaying information visually through a map, the list view provides similar information, but in a sortable, filterable table.

Stimulus/Response Sequences

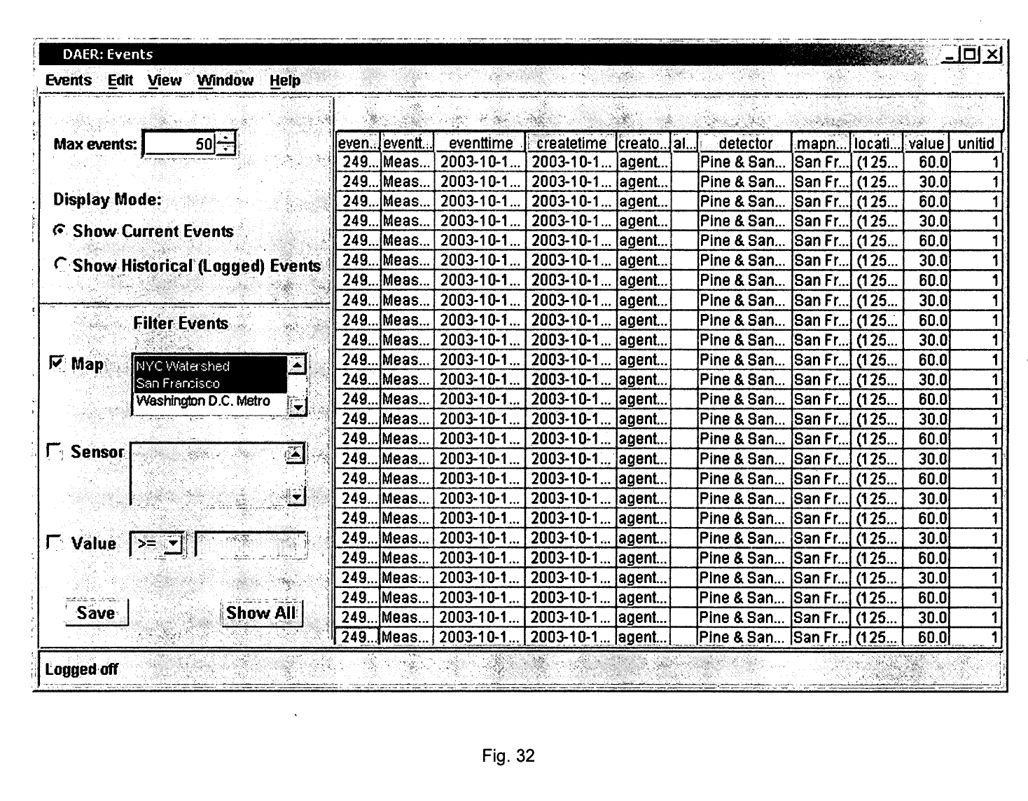

As with the Map view, the List view is a window that is accessed through the Client application. The type and amount of information that is available depends on the user's selection(s). FIG. 32 is a screen shot of a user interface illustrating the primary functionality which this window should have.

Functional Specifications

SPEC-1: This table should list all the selected location's map objects (i.e., all sensors and all agents).

SPEC-2: The table should be sortable by any column. Sorting occurs by clicking on the column's title (1 click sorts descending; another click sorts it ascending). Subsequent sorting-within-sorting should be done by holding down the control key (or other mechanism) before selecting a column.

SPEC-3: Users should be able to arrange the column order, moving different columns into different positions in the table. Columns should be moved by clicking and holding on a column title, then dragging it to where it should be placed. (An alternative method could be an “Arrange” button that brings up a dialog with a list of column names that you can move up/down to reorder the table)

SPEC-4: Because of the potentially large amount of information, this table should have a filter mechanism to reduce the number of entries. The filter should be robust enough to allow rather involved filtering (such as, list only those sensors which are alert status, radiological type, and which lie within a certain defined network or region).

SPEC-5: Columns to be included are:
Icon (e.g., green sphere)
ID (445234)
Name (SFO Radiological #234)
Type (radiological, biological, chemical, heat, HazMat, Red Cross, etc.)
Location
Profile
Sensor Network (as defined by a specific set of sensors)
Sensor Status (normal, alert, offline)

SPEC-6: Rolling the mouse over any part of a row that represents a sensor should cause a popup window to appear with the sensor's ID, name, condition (normal, alert, offline) description, and most recent measurements.

SPEC-7: Double-clicking any part of a row that represents a sensor should open the sensor's configuration dialog.

SPEC-8: Rolling the mouse over any part of a row that represents a responder should cause a popup window to appear with the responder's ID, name, description, and most recent location.

SPEC-9: Double-clicking any part of a row that represents a responder should open the responder's configuration dialog.

SPEC-10: Entering a number in Max Events will limit the number of visible rows displayed.

SPEC-11: Provide an option to either stream real-time events, or instead, to load a chunk of historical, logged events.

Personnel & Mobile Device Tracking

Description

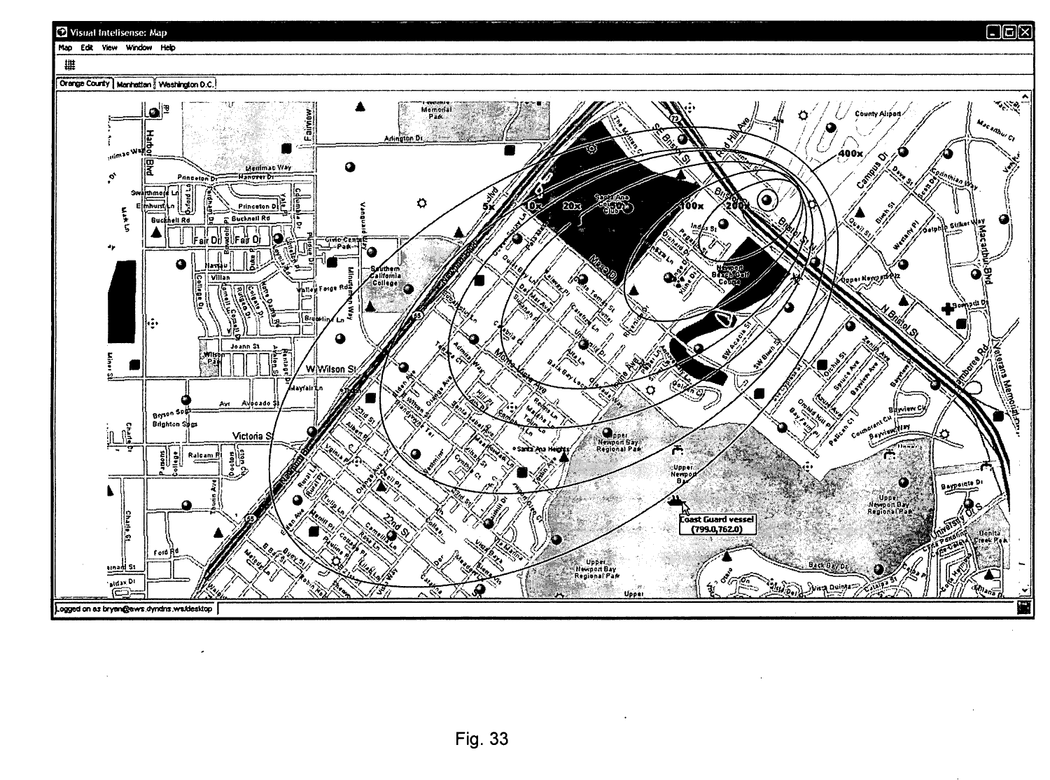

Aside from detecting and tracking the spread of a radiation cloud or other emergency event, the product will preferably possess the ability to track personnel and/or other mobile devices. This feature leverages the Map View capability, and loads the mobile objects (i.e., people and devices) into respective Overlays in the manner illustrated by FIG. 33.

Stimulus/Response Sequences

Positioning information is provided by GPS-enabled network motes, which send their position data along with other information to the Server. The Server then processes the position information and sends the updated details to the Client.

Functional Specifications

SPEC-1: Should display icons for responders. Each different type of responder should have a different symbol. The system should update each responder's position as changes in its location data are detected.

SPEC-2: An object whose location is identified as having moved outside the scope of the active map should be removed from the map. Movement data concerning all objects should sill be logged, however. Any time an object is determined to have moved into the selected map's region, it should be overlaid on the map (assuming that overlay is active).

SPEC-3: Rolling the mouse over a responder icon should cause a popup window to appear with the responder's ID, name, description, and most recent location. Double-clicking a responder icon should open the responder's configuration dialog.

Sensor Profiles

Description

Sensor Profiles greatly reduce the effort of manually controlling hundreds or thousands of sensors. Profiles let users group a specified set of the same type of sensors. Different types of profiles let you mix and match a variety of different sensors (based on sensor type, location, etc.), to obtain a very granular level of control and specificity.

Stimulus/Response Sequences

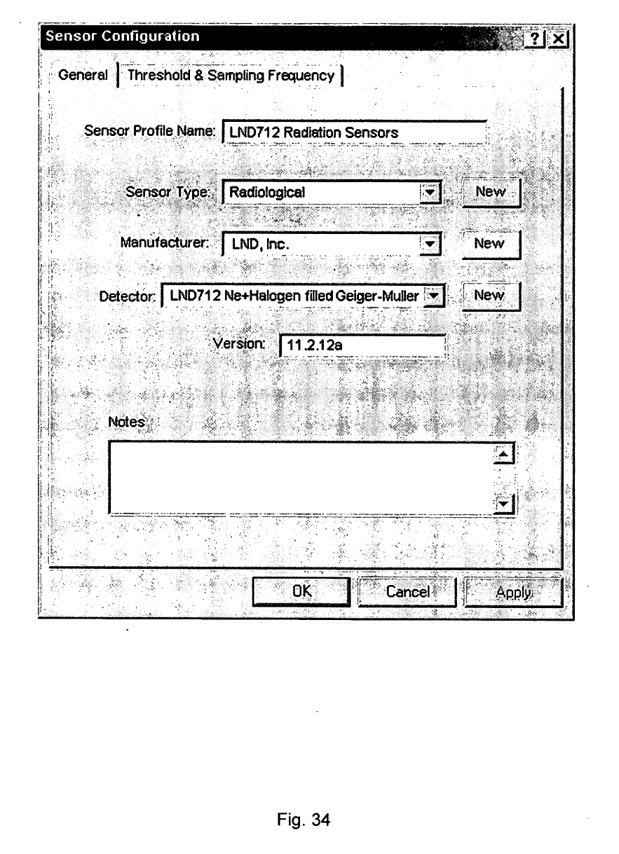

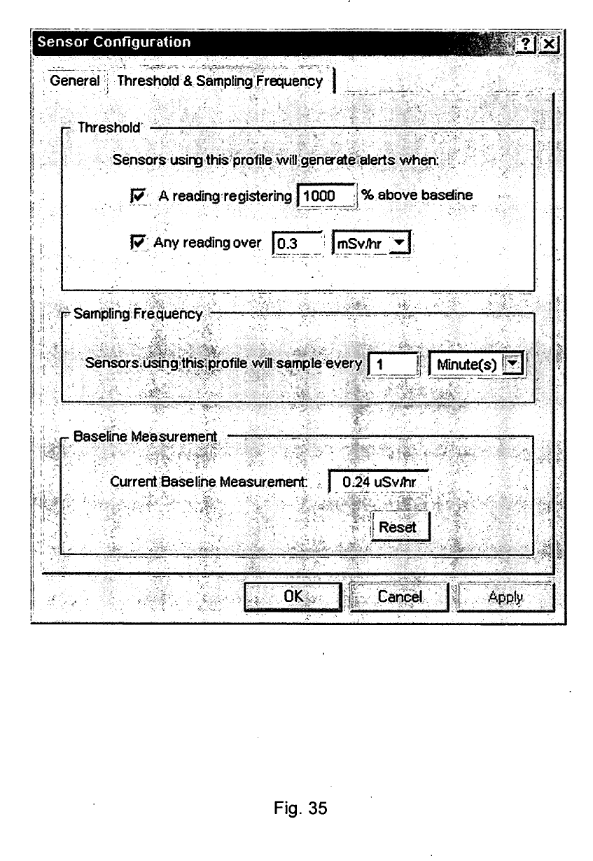

Defining a sensor profile is done through the Client application, as is illustrated by the screen shots of FIGS. 34-35.

Functional Specifications

SPEC-1: Sensor Name: Users should be able to name the sensor, independent of the unique ID the system allocates for it.

SPEC-2: Sensor Type: There should be a mechanism to specify the type of sensor, along with an edit function to add or delete to/from the type list.

SPEC-3: Manufacturer: This field would probably be better served being titled “Maintenance Provider,” as the idea is to provide a reference to a company POC who has an SLA with the client company. If/when a malfunction is detected in this particular sensor, this field would provide a lookup to the proper contact info. The Server would then email the contact, notifying them of the need to repair/replace, providing specific location information, as well as log details that describe the nature of the malfunction. There should also be an edit function to allow users to add new contractors and modify and/or delete existing ones.

SPEC-4: Detector: There should be a field where the user can identify the specific type of detector that is contained in the sensor package. This information needs to be granular enough to identify brand name and model. As with other fields, this one requires an edit function to define new detectors as well as edit/delete ones no longer used.

SPEC-5: Version: The editable version field is to describe a particular version of a given detector model.

SPEC-6: Notes: This editable field lets the user provide any additional information that is relevant to the sensor. In particular, the location of the device, date last serviced, whether it's near a naturally high radiation source, etc.

SPEC-7: Threshold (group): Each sensor profile should have a defined threshold level. Threshold values and types will depend on the type of sensor.

SPEC-8: Sampling Frequency: This is another required field that the user should complete before saving the sensor profile. It controls how frequently the sensor takes readings. What remains to be designed is a more complicated version that allows for change to the rate when a threshold level is reached.

SPEC-9: Reporting Frequency: This concept is similar to the Sampling Frequency, but it controls how frequently the sensor transmits its collected data. By definition, the reporting frequency can be no shorter a time period than the sampling frequency. This item should also have at least two values: a normal condition frequency, and an accelerated rate when a threshold level is detected.

SPEC-10: Baseline: The baseline entry may be where the user could manipulate the baseline parameters, such as type of moving average, length of the average, etc.

Region Profiles

Description

Region profiles group any number and type of sensors that are to be defined as within a defined geological neighborhood. This profile can be used to identify a large number of sensors for test purposes, etc.

Example: “NYC Profile” identifies all the different sensors that fall within metro New York City.

Stimulus/Response Sequences

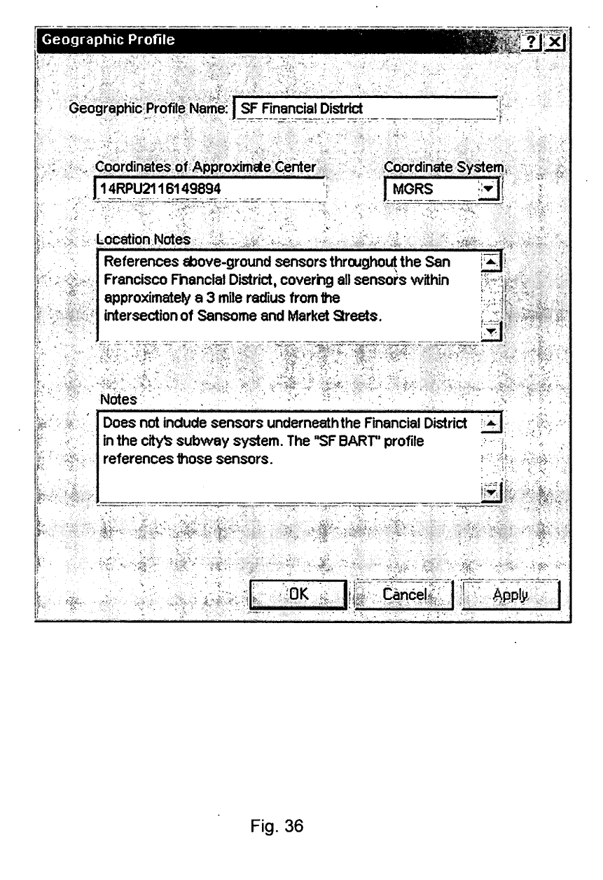

Defining a region profile is done through the Client application in the manner illustrated by the screen shot of FIG. 36.

Functional Specifications

SPEC-1: Profile Name: Required to uniquely identify this profile.

SPEC-2: Approximate Coordinates: Need some identifiable central point that can be used to programmatically specify a group of sensors within a given range. For example, giving a coordinate, along with a 1-mile radius can quickly define. all the different sensors whose cords fall within this defined area.

SPEC-3: Notes: Provide an editable area where the user can enter comments, descriptions of the geography, or what may be more important, areas of exclusion.

Scenario Profiles

Description

This profile is actually a “meta-profile” that uses Sensor Profiles and Region Profiles to build a powerful response-enabling device.

Scenario Profiles let emergency personnel pre-define a particular scenario and alert the appropriate responders and automatically send activation messages to other emergency responder systems.

Example: The “WashDC Subway-Chem” profile determines what processes should be activated when a chemical agent is detected in the Washington DC subway. Because the system has already defined a “WashDC Subway” Region profile as all sensors within the Washington DC subway system, and “Chem” specifies a chemical alert, you can then specify which people and automated systems should receive this information and act accordingly, such as notifying the metro transit authority and potentially sending a pre-defined signal to the right system to turn off the subway's HVAC system.

Stimulus/Response Sequences

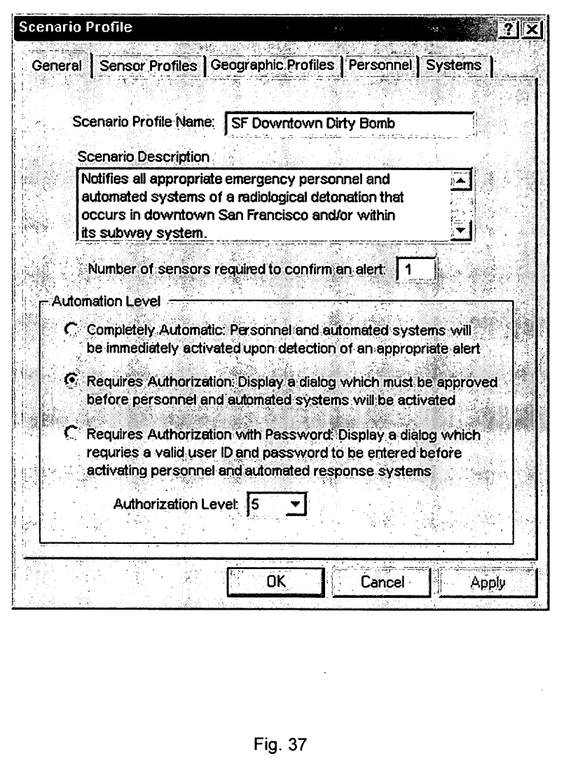

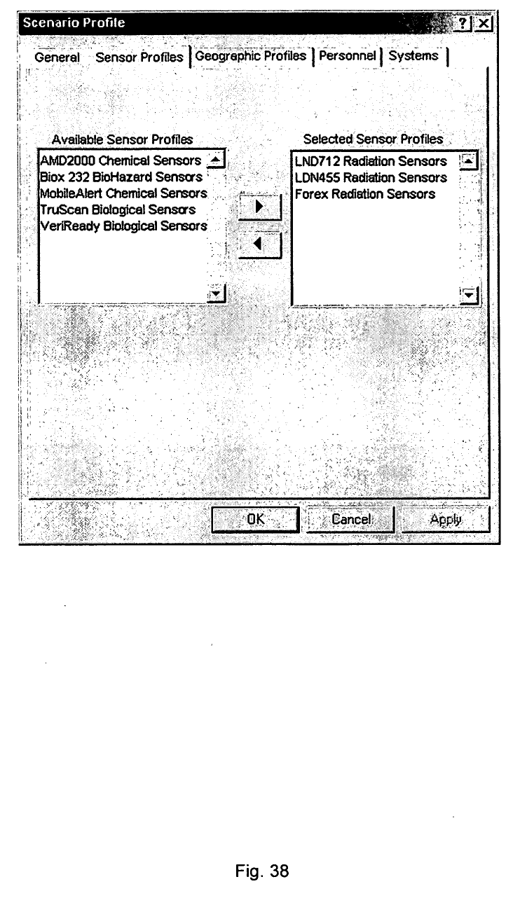

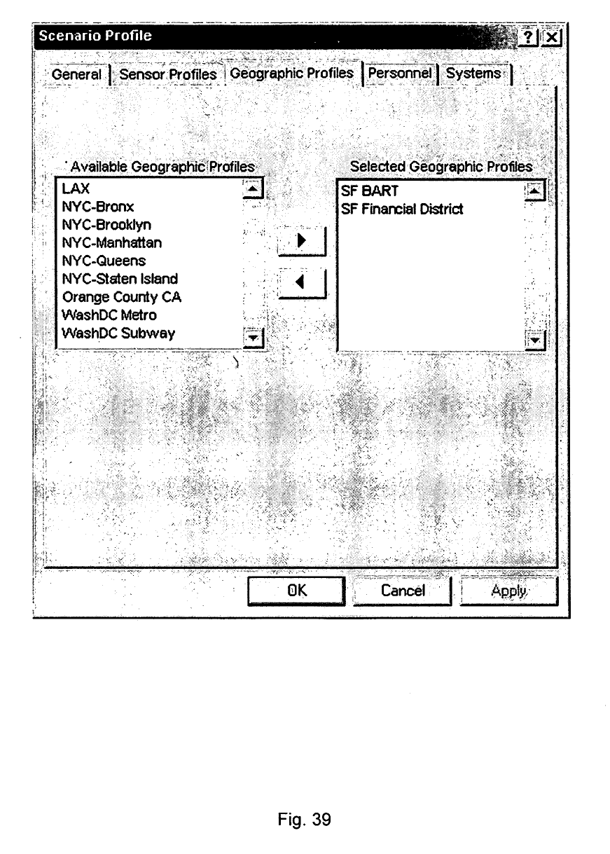

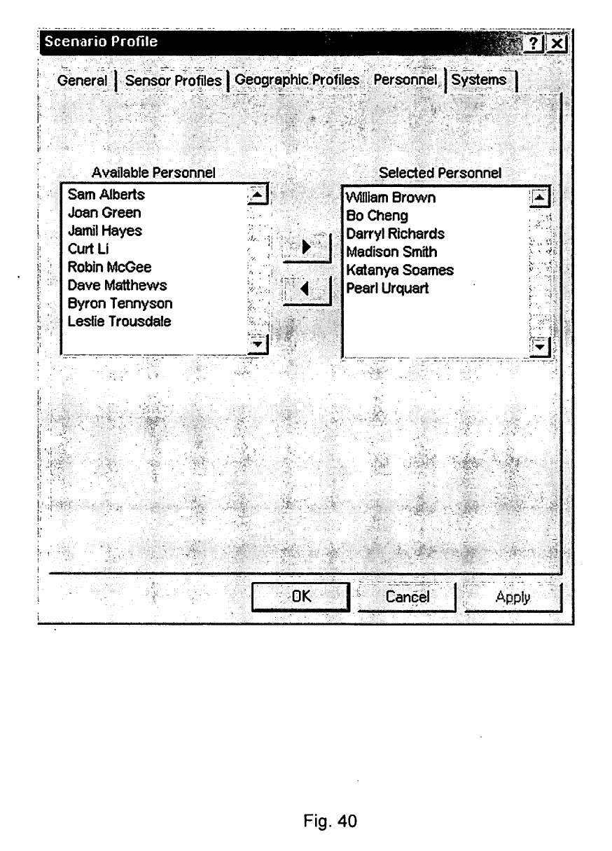

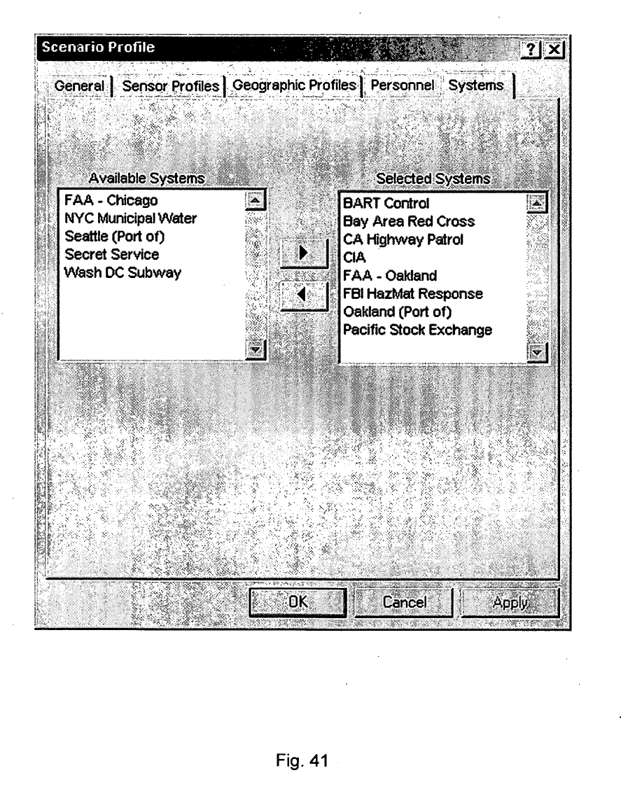

Defining a scenario profile is done through the Client application in the manner illustrated by the screen shots of FIGS. 37-41.

Functional Specifications

SPEC-1: Profile Name: Required to uniquely identify this profile.

SPEC-2: Description: Allow an editable field for the user to clearly describe the scope of this scenario profile.

SPEC-3: Number of Sensors: Minimize false alarms by requiring more than one sensor in a given area to detect threshold events before activating this sensor. This may be achieved by providing a preference setting permitting specification of the maximum distance “X” between sensors within the same given area. The actual distance between sensors is either calculated by known, fixed locations, or by calculating the latest positing information streaming from a GPS-enabled mobile device.

SPEC-4: Automation Levels: Provide at least three levels of automated response to the scenario alert: at the most basic, have the system immediately notify all defined users and active all specified systems. Level 2 would first provide a dialog box that would request a user to accept or decline to activate the system. Level 3 would not only display a dialog, but would also require the user to enter a username and password. The user ID authority level should be the same level or higher as what is stipulated in the scenario profile definition.

SPEC-5: Should be able to select a pre-defined sensor profile. An additional benefit would be to enable multiple profiles to be selected, but this would also entail more complicated AND OR capabilities to be truly valuable.

SPEC-6: Should be able to select a pre-defined region profile.

SPEC-7: Should be able to select the personnel (who are pre-defined users in the system) to be notified.

SPEC-8: Should be able to select the appropriate automated systems to automate (which have been pre-defined from the integration).

Maintenance Profiles

Description

Maintenance profiles take advantage of the system's ability to monitor its own health. If a particular sensor fails to provide a heartbeat, or shows other signs of a malfunction, a maintenance profile is used to automatically identify the SLA contractor responsible for repairs and send them an alert to repair or replace the sensor.

Stimulus/Response Sequences

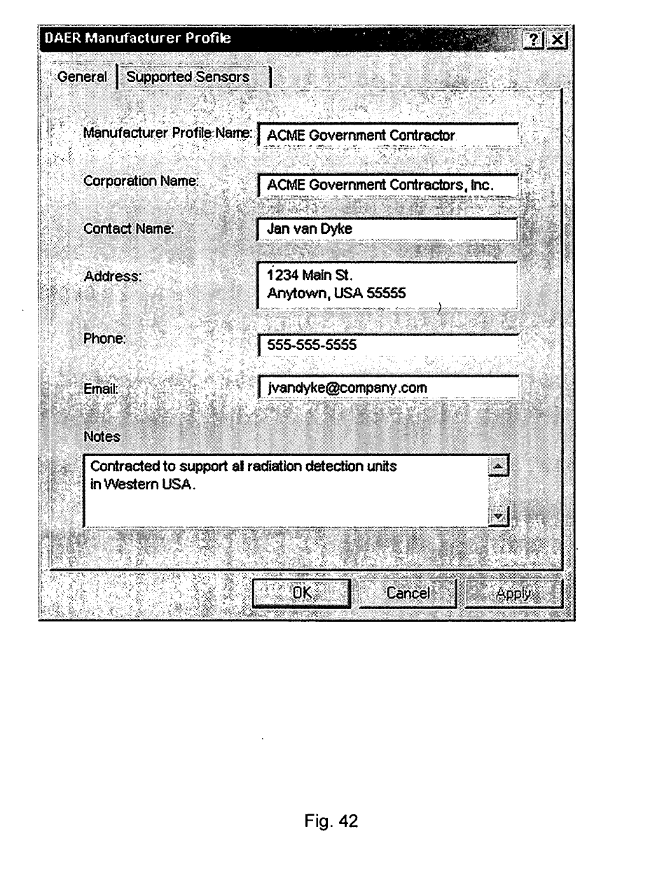

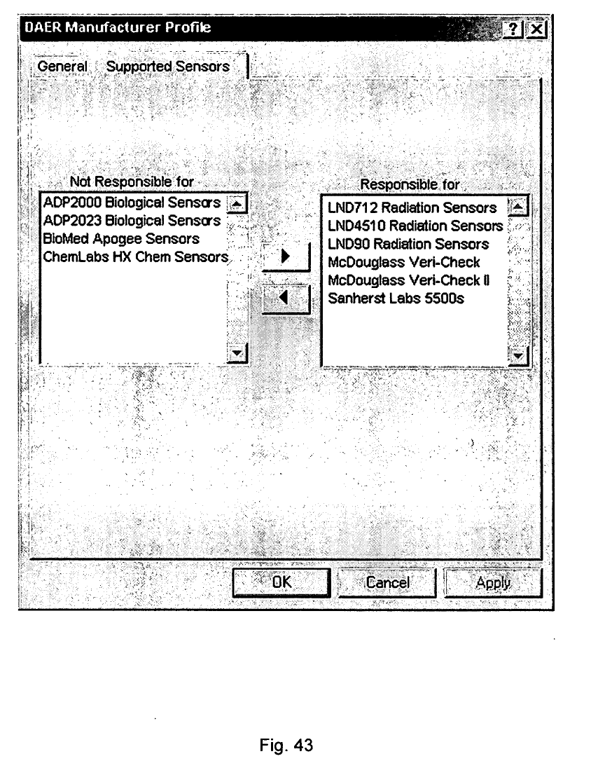

Defining a maintenance profile is done through the Client application in the manner illustrated by the screen shot of FIGS. 42-43.

Functional Specifications

SPEC-1: Profile Name: Required to uniquely identify this profile.

SPEC-2: Contact Info: Should provide fields to enter all relevant contact information, including company name, POC, phone number, email, etc.

SPEC-3: Notification: System should use email (and possibly other communication channels) to automatically notify service provider of problem and provide details.

SPEC-3: Notes: Should have an editable field where user can enter information concerning the nature of the contract/SLA, etc.

SPEC-4: Supported Sensors: Should be able to leverage Sensor Profiles and Region Profiles to specify both what types of sensors this profile covers, but also within which geographic area.

User Management

Description

Users should be defined within the system in order to log on and gain access. Different levels of authority will also dictate what information they can see. The prototype supports all its user and group information internally, however in commercial deployments, we expect to access corporate directories.

Stimulus/Response Sequences

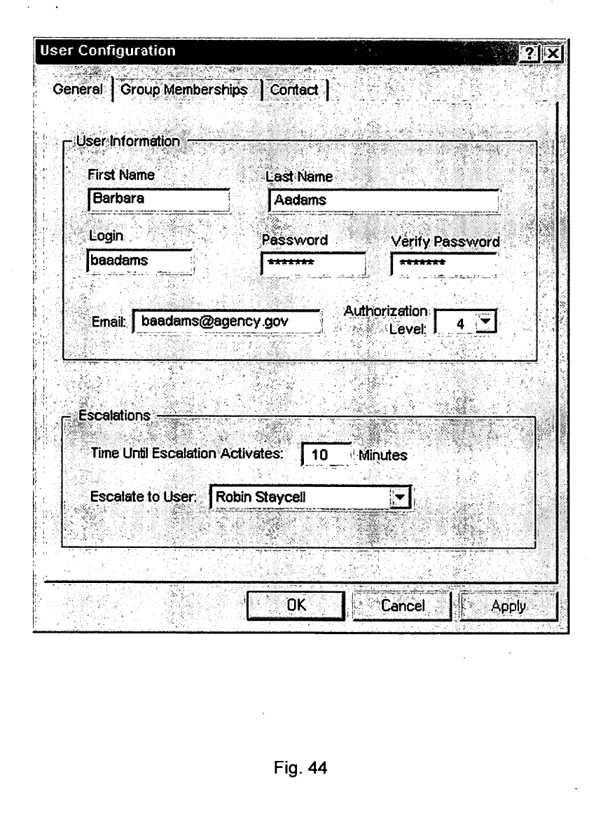

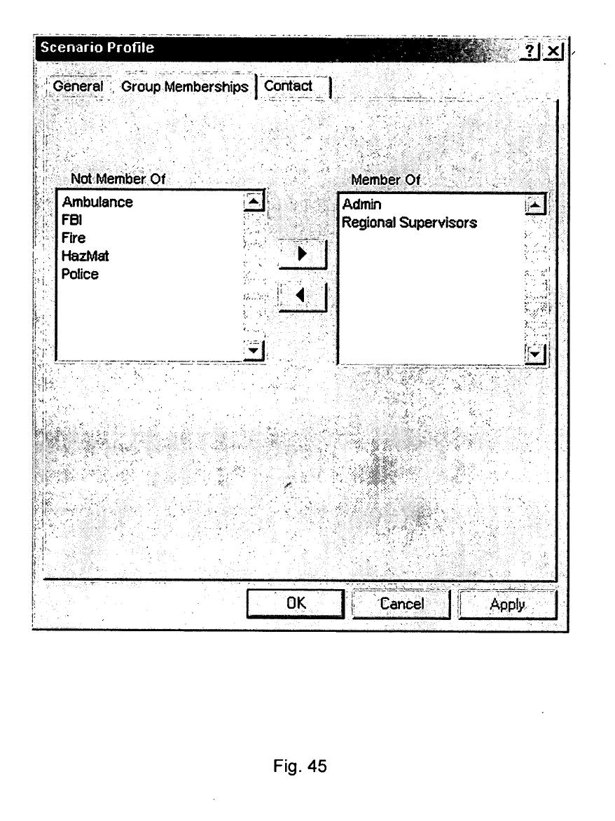

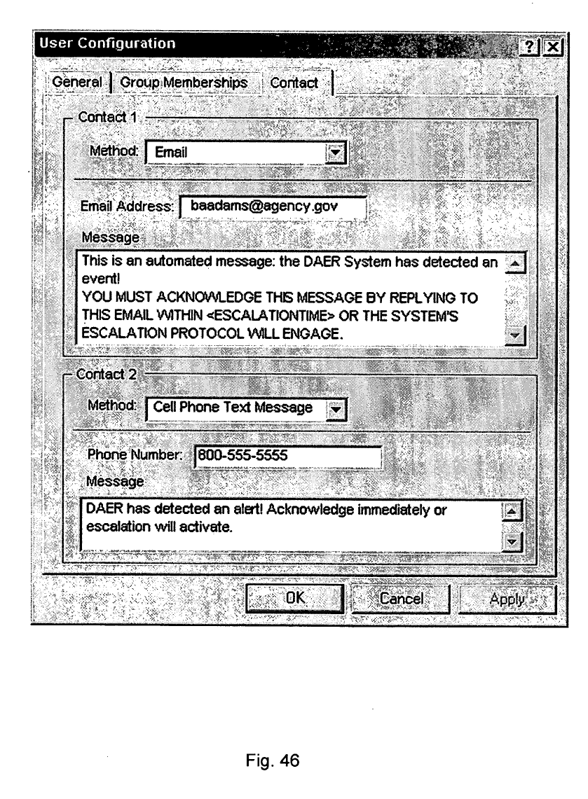

Defining a user is done through the Client application in the manner illustrated by the screen shots of FIGS. 44-46.

Functional Specifications

SPEC-1: User Information: Aside from an internally generated unique ID, each user will be uniquely identified by their user information. Fields should support all the standard user info, including full name, user ID and password, authorization level, and contact information.

SPEC-2: Escalations: We should create an escalation engine that passes the same notification information about an emergency up through the chain of command if the user doesn't respond within a set period of time.

SPEC-3: Group Memberships: Provide a mechanism by which this user can be added to, or removed from a set of pre-defined groups.

SPEC-4: Contact Preferences: Design an area that supports a variety of communication channels. Support at minimum email (SMTP, which is already built into the prototype); additional messages to support could be SMS, pager, etc. By defining these in a specific order, the system knows which mechanism to use to attempt first contact, which to use as backup, etc.

SPEC-5: Variable support in messages: Users should be able to define “template” messages (e.g., for email) where they can enter variables that, when parsed by the Server, will be converted to standard text before being sent. Examples include <escalation_time>, <scenario_profile>, <emergency details>, etc.

Group Management

Description

Similar to users, groups should be defined within the system in order to organize users into functional sets. Different groups could have access to different parts of the system, etc. The prototype supports all its user and group information internally, however in commercial deployments, we expect to access corporate directories.

Stimulus/Response Sequences

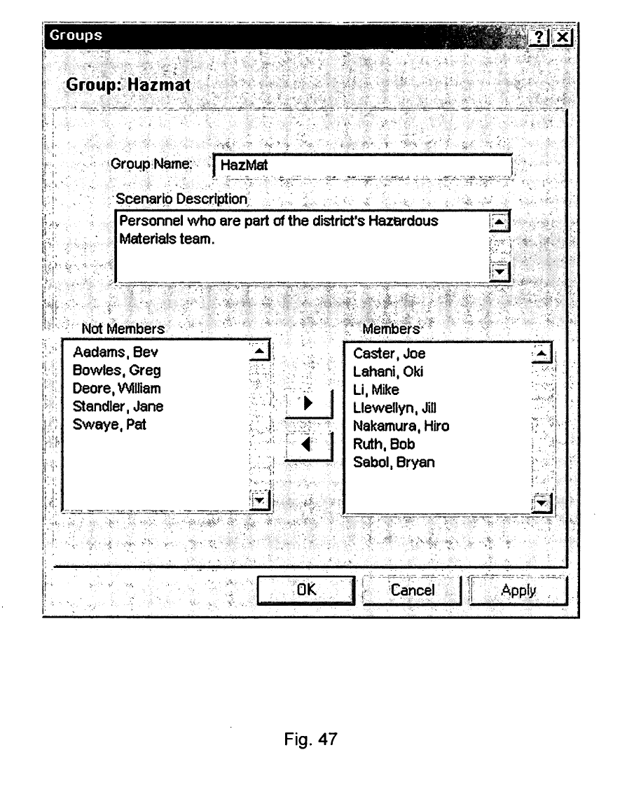

Defining a group is done through the Client application in the manner illustrated by the screen shot of FIG. 47.

Functional Specifications

SPEC-1: Name and Description: Aside from the internally generated group ID, a unique group name will be used to identify this group. A description field should also be provided to

SPEC-2: Members: Populate the set of pre-defined users and provide a mechanism to specify which will be associate with this group.

SPEC-3: Access: It may be desired to appoint a group to have access to a particular area within the system. Example: only members of the group “Admin” may access the admin screens.

Backups and Archiving

Description

The system constantly records data from a multitude of networked sensors and other objects. This information should have a backup and archival mechanism that allows the system to automatically offload its log data and configuration settings to a networked backup system.

Stimulus/Response Sequences

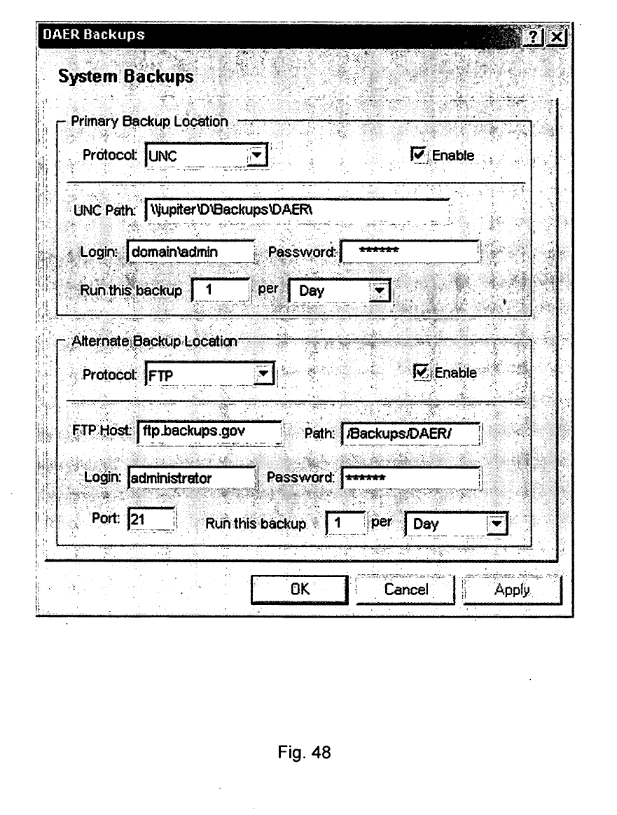

Setting system backups is done through the Client application in the manner illustrated by the screen shot of FIG. 48.

Functional Specifications

SPEC-1: Specify location to dump backups, along with required access information.

SPEC-2: Provide a mechanism that lets admins choose what items are to be backed up

SPEC-3: Aside from scheduling capabilities, should also provide a manual “Do It” button to immediately run backup.

System Logs

Description

The system should log its activities. By offering a variety of levels of logging (i.e., high level vs. trace, etc.), administrators can balance the amount of detail they want to have vs. required the storage space.

Stimulus/Response Sequences

Setting log preferences is done through the Client application.

Functional Specifications

SPEC-1: Should let admin set level of log detail (high level, trace, etc.)

SPEC-2: Log sensor details (complete, or alert-level only)

SPEC-3 Log system status details (system health, malfunctioning sensors, etc.)

External Interface Specifications

Hardware Interfaces

Detector-Mote Interface

Functional Specifications

SPEC-1: Wireless mote should be able to function within the mesh network, performing the tasks normally associated with mesh-networks, such as self-organizing, passing data to other motes and/or receiving and transmitting data from other more distant motes to the gateway, etc.

SPEC-2: The interface should support 2-way communication so that not only can the sensor pass data to the Server, but the Server can communicate with a particular sensor in the network to change various parameters (e.g., modify sampling rate, or frequency of transmission, or possibly to stop transmitting entirely due to identified malfunction, etc.).

SPEC-3: The sensor should support three different modes of operation:
Interval-Based: This is the default behavior for non-emergency situations which is designed to conserve power. In interval-based mode, the sensor samples the environment on a regular basis (e.g., every 5 minutes) and saves the value. Then, once every defined longer period (e.g., every 15 minutes), the sensor transmits the collected data packet.
Event-Based: This mode is controlled by events, such that some event like registering threshold level ambient data will result in accelerated sampling and broadcasting rates (e.g., sample every second and broadcast every 5 seconds).
Mixed-Mode: This is the preferred mode that allows the combination of the two previously described modes. In this situation, the same sensor typically operates under interval-based mode until a threshold value is obtained (or an event is sent to it by the Server). This received event switches the behavior to event-based mode

SPEC 4: The sensor will preferably be housed in a weatherproof container and provided appropriate transmission hardware (e.g., external antenna) to allow a suitable signal to be sent and received by other mesh motes.

SPEC-5: The sensor should be able to send a “heartbeat” through the network to confirm its continuing operation.

SPEC-6: The communication between the sensor and the Server should be secure at both the packet and network level (i.e., encrypted data running over an encrypted wireless network).

Software Interfaces

Events and Data Flow

Events are things that have occurred in the system. By definition, once an event has occurred it does not change. There is a set of predefined events. These events are created internally as the system runs.

Users may define their own events following the general schema listed below:

| Attribute | Description |
|---|---|
| id | Unique id |
| datetime | Date and time in GMT of the event. |
| type | Type code of the event. |
| creator | ID of the event originator. |

Specific event types may have additional attributes. For example, the logon event has a user name and IP address.

Sensor Events

An important event is the sensor event. This event is a detector measurement at a specific time. Sensor events are expected to be continuously created by the sensor. This requirement provides a heartbeat mechanism that allows the system to determine whether a sensor is operational. It also allows detailed history to be stored. Lastly, it reduces the complexity of the sensor.

Table I describes the sensor event.

TABLE I

| Attribute | Description |
|---|---|
| id | Unique id |
| datetime | Date and time in GMT of the event. |
| type | Always 1. |
| creator | ID of the event originator. This is the sensor agent's |
| | unique id set at installation. |
| detector | ID of the detector. This is unique for the sensor. A |
| | globally unique id for the detector is created by using |
| | the sensor id and the detector id. |
| location | The attribute is optional. Fixed detectors location is |
| | already known. This attribute is for mobile detectors |
| | that can report their location via GPS or some other |
| | means. Value format to be determined. |
| units | The type of measurement. 1 = microSieverts (radiation) |
| value | The measurement value. |
| accuracy | accuracy of the measurement in units. This is expected |
| | to be a constant value for most detectors but may vary |
| | for others. |

Communications Interfaces

Sensors may communicate to the message handler daemon by streaming XML on a TCP socket using the XMPP protocol. This connection normally uses SSL 2.0 (server authentication). SSL 3.0 (client authentication) can also be used but no server side client certificate authentication is done.

Glossary

EOC: See Emergency Operations Center.

Emergency Operations Center. This is both a physical location as well as an assemblage of public officials who gather at the emergency facility to coordinate response to an emergency.

Intelisense Client: The software application that provides the GUI to monitor and interact with the system.

Intelisense Server. The back-end part of the system which receives sensor data, analyzes their values, posts map and object information to the Clients to view, and interfaces with other communication channels, etc., in the client's infrastructure.

Maintenance Profile: A definition that specifies a particular contractor (typically operating under some SLA) who is responsible for ongoing maintenance and repair of one or more types of sensors in the sensor network. The system uses the profile to automatically look up the appropriate contact info and send a maintenance alert to the contractor when a malfunction is detected.

Mesh Network: A newer wireless network methodology which includes battery-operated wireless radio “motes” that can “self-organize” into a viable communication network. This self-organizing and self-healing nature of a mesh network is what differentiates it from traditional wireless networks.

Region Profile: A definition that specifies all the different sensors within a certain defined geographic area. This information can be used in several ways: to identify the appropriate maintenance contractor who is responsible for a malfunctioning sensor in a particular area; to determine which scenario profile to activate, based on the location of the emergency, etc.

Scenario Profile: A definition that leverages both sensor profiles and region profiles, and ties their information together with users and automated systems. It is the scenario profile that gives VI the power to immediately notify the correct set of people and activate the right automated systems when a dirty bomb is detected in an amusement park, vs. a toxic spill occurring on a freeway outside of a major metro area.

Sensor: A sensor is a combination of a detection device (e.g., radiation, air temp, etc.), along with a communication component (e.g., a wireless network mote or wireline network interface) that allows the detector to stream its ambient data to the Intelisense Server.

Sensor Profile: A definition that describes one particular kind of sensor (e.g., an LND712 radiation detector). The profile will allow the user to set key sampling frequency and threshold levels within the profile, which can then be used to control a large number of these sensors in a given sensor network.

VI. See Virtual Intelisense.

Virtual Intelisense (Module): A collection of scripts used to generate simulated, or “virtual” sensor data.

The foregoing description, for purposes of explanation, used specific nomenclature to provide a thorough understanding of the invention. However, it will be apparent to one skilled in the art that the specific details are not required in order to practice the invention. In other instances, well-known circuits and devices are shown in block diagram form in order to avoid unnecessary distraction from the underlying invention. Thus, the foregoing descriptions of specific embodiments of the present invention are presented for purposes of illustration and description. They are not intended to be exhaustive or to limit the invention to the precise forms disclosed, obviously many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated. It is intended that the following Claims and their equivalents define the scope of the invention.

What is claimed is:

1. A system for detecting an event having environmental consequences, the system including:
a plurality of sensors capable of detecting one or more environmental agents;
a plurality of sensor modules, wherein each of the plurality of sensor modules is capable of receiving data produced by at least one of the plurality of sensors; and
a central server in communication with one or more of the plurality of sensor modules, the central server being configured to generate an alert indicative of occurrence of the event based upon information received from the one or more of the plurality of sensor modules.

2. The system of claim 1 wherein each of the sensor modules is capable of communicating with other of the sensor modules.

3. The system of claim 1 wherein the environmental agents include chemical, radiation or biological agents.

4. The system of claim 1 wherein the central server is capable of controlling one or more parameters relating to transmission of the information from the one or more of the plurality of sensor modules to the central server.

5. The system of claim 1 wherein the central server is capable of generating user interface information relating to representation of the environmental consequences of the event.

6. The system of claim 5 wherein the user interface information includes status information pertaining to ones of the plurality of sensors.

7. The system of claim 6 wherein the status information includes historical sensor data.

8. The system of claim 7 wherein the central server includes a visualization component capable of generating display information from which a visual representation of the historical sensor data may be rendered.

9. The method of claim 1 wherein the central server includes means for recommending a response to the occurrence of the event.

10. A method monitoring an environment for threat conditions potentially related to a catastrophic event is disclosed herein, the method comprising:
determining baseline levels of one or more environmental agents in an area of the environment based upon first sensor measurement data;
establishing one or more threshold levels relative to the baseline levels;
processing second sensor measurement data with respect to the one or more baseline levels;
identifying at least one threat based upon the processing of the second sensor measurement data; and
assessing the at least one threat.

11. The method of claim 10 further including assigning a priority to the at least one threat.

12. The method of claim 10 further including recommending a response based upon the at least one threat.

13. The method of claim 10 wherein the assessing the at least one threat is performed with reference to at least one predefined profile associated with at least one of the plurality of sensors.

14. The method of claim 11 wherein the assigning a priority includes establishing a plurality of predefined priority values and assigning one of these plurality of predefined priority values to the at least one threat.

15. The method of claim 12 wherein the recommending a response includes:
associating a recommended response with each of a plurality of scenario profiles, and
determining an applicable one of the plurality of scenario profiles and identifying the associated recommended response.
